(19) United States
(12) Patent Application Publication
(10) Pub. No.: US 2007/0149978 A1
(43) Pub. Date: Jun. 28, 2007

(54) Minimally invasive distraction device and method

(76) Inventors: Yuval Shezifi, Haifa (IL); Ilan Grunberg, Haifa (IL); Nissim Ohana, Ranana (IL); Asaf Ben-Arye, Zichron Yaakov (IL)

Correspondence Address: SALTAMAR INNOVATIONS, 30 FERN LANE, SOUTH PORTLAND, ME, 04106, US

(21) Appl. No.: 10/551,056

(22) PCT Filed: Feb. 22, 2004
(86) PCT No.: PCT/IL04/00171

§ 371 Date: Feb. 7, 2007

(30) Foreign Application Priority Data
Mar. 30, 2003 (IL) 155146

(51) Int. Cl. A61B 17/58

(52) U.S. Cl. 606/90

(57) ABSTRACT

A device (10) for distracting and supporting two substantially opposing tissue surfaces in a patient's body, to be introduced within the tissue surfaces in a minimally invasive procedure. The device comprises: a wrapping element (12); and an expandable structure (24) insertable between the two substantially opposing support surfaces of the wrapping element, adapted to be expanded between the two substantially opposing surfaces to a predetermined dimension.

### FIELD OF THE INVENTION

The present invention relates to tissue distraction and support. More particularly, it relates to a device and method for minimally invasive distraction and support device, which is introduced within two tissue surfaces and provides distraction and support.

### BACKGROUND OF THE INVENTION

The spinal column serves as the support structure of the body, rendering the body its posture. Yet age, diseases and traumas hamper its completeness, and health, causing structural failures such as vertebral fractures, disc hernias, degenerative disk diseases, etc., resulting in pain and spinal instability, and even paralysis.

Among various vertebral column disorders, the typical ones include traumatic damages such as compression fractures, degenerative disc disease, disc hernias (ruptured or protruded disc), scoliosis (lateral bending of the vertebral column), kyphosis (exaggerated thoracic curvature), lordosis (exaggerated lumbar curvature), and spina bifidia (congenital incompletion of the closure of the vertebral column).

Distraction may also be considered in Tibial Plateau, a compression fracture in which the articular surface of the condyle (one or both) depresses. The treatment involves elevating or distracting the compressed surfaces to reduce the fracture. Once aligned, filler is inserted into the void to maintain the distance. No screws or nails are used to stabilize the bone.

Another example for the need for distraction is in Spinal Interbody Fusion. In the case of chronic back pain, usually due to the presence of an unstable disc (degenerative or trauma related), removal of the disc is usually a common treatment. To decrease pain spinal motion at the injured area is reduced. Fusion of the two vertebrae can be achieved by removal of some of the defective disc and insertion of some bone graft that assists bone fusion of the two adjacent vertebrae. Another common practice involves inserting metallic implants to further stabilize the vertebrae. In addition, screws, plates, hooks, and rods are also used in the procedure. Placement of the fixed height implants in between the two adjacent vertebrae requires districting them from one another.

Various distraction, fixation, replacement and reconstructive solutions for distraction, replacement, and reconstruction—both intravertebral and intervertebral were introduced in the past, some of which are mentioned hereinafter.

For example, U.S. Pat. No. 6,019,793 (Perren et al.), titled SURGICAL PROSTHETIC DEVICE, disclosed a surgical prosthetic device that is adapted for placement between two adjoining vertebrae for total or partial replacement of the disk from therebetween. The device has two plates with interior surfaces facing each other and being held at a distance by connecting means and exterior surfaces for contacting the end plates of the two adjoining vertebrae. The connecting means is made of a shape-memory alloy so that it is delivered to its destination squashed within a delivering tool and deploys once freed in position.

U.S. Pat. No. 5,423,816 (Lin) titled INTERVERTEBRAL LOCKING DEVICE disclosed an intervertebral locking device comprising one spiral elastic body, two bracing mounts and two sets of locking members. The two bracing mounts are fastened respectively to both ends of the spiral elastic body. The two sets of locking members are fastened respectively with the two bracing mounts such that each set of the locking members is anchored in one of the two vertebrae adjacent to a vertebra under treatment. The spiral elastic body and the vertebra under treatment evince similar elastic qualities, i.e. similar deflection characteristics. A plurality of bone grafts affinitive to the vertebra under treatment is deposited in the chambers of the spiral elastic body and in the spaces surrounding the spiral elastic body.

U.S. Pat. No. 5,423,817 (Lin) titled INTERVERTEBRAL FUSING DEVICE, teaches an intervertebral fusing device having a spring body portion interconnecting a first spiral ring mount and a second spiral ring mount. Each spiral ring mount has a spiralling projection on the outer surface. The spring body portion is defined by a plurality of spiral loops. The plurality of spiral loops and spiralling projection of the spiral ring mounts have a constant pitch. A mount cover and a head member are threaded into an internally threaded portion of a respective spiral ring mount thereby forming a chamber in which bone grafts affinitive to the cells and tissues of a vertebra may be housed. The spring body portion is similar in elasticity to the vertebra.

U.S. Pat. No. 5,306,310 (Siebels), titled VERTEBRAL PROSTHESIS, disclosed a prosthesis as a vertebral replacement element consisting of two helical strands, which may be screwed together to form a tubular structure. The implant is inserted between vertebrae and then slightly unscrewed until the desired height is reached. The helical strands consist of carbon fiber reinforced composite material.

U.S. Pat. No. 6,033,406 (Mathews) titled METHOD FOR SUBCUTANEOUS SUPRAFASCIAL PEDICULAR INTERNAL FIXATION disclosed a method for internal fixation of vertebra of the spine to facilitate graft fusion includes steps for excising the nucleus of an affected disc, preparing a bone graft, instrumenting the vertebrae for fixation, and introducing the bone graft into the resected nuclear space. Disc resection is conducted through two portals through the annulus, with one portal supporting resection instruments and the other supporting a viewing device. The fixation hardware is inserted through small incisions aligned with each pedicle to be instrumented. The hardware includes bone screws, fixation plates, engagement nuts, and linking members. In an important aspect of the method, the fixation plates, engagement nuts and linking members are supported suprafascially but subcutaneously so that the fascia and muscle tissue are not damaged. The bone screw is configured to support the fixation hardware above the fascia. In a further aspect of the invention, a three-component dilator system is provided for use during the bone screw implantation steps of the method.

Generally, these described methods and devices are very invasive and involve massive surgical involvement.

Minimally invasive system is described in U.S. Pat. No. 6,248,110 (Reiley et al.) titled SYSTEMS AND METHODS FOR TREATING FRACTURED OR DISEASED BONE USING EXPANDABLE BODIES. Systems and methods are disclosed for treating fractured or diseased bone by deploying more than a single therapeutic tool into the bone. In one arrangement, the systems and methods deploy an expandable body in association with a bone cement nozzle into the bone, such that both occupy the bone interior at the same time. In another arrangement, the systems and methods deploy multiple expandable bodies, which occupy the bone interior volume simultaneously. Expansion of the bodies forms cavity or cavities in cancellous bone in the interior bone volume. Use of expandable balloon is taught, which serves for reconstruction of collapsed bone. In order to fill the space created and provide stabilization to the bone, insertion of polymethylmethacrylate cement that hardens and stiffens is required.

US 2002/0183761 (Johnson et al.) disclosed an apparatus and method for distracting, in a given direction, and supporting two tissue surfaces. A plurality of wafers is consecutively inserted between the two tissue surfaces to create a column of wafers. The column of wafers is oriented between the tissue surfaces so as to expand in the given direction as the wafers are consecutively added to the column. An introduction device was described in the form of a tube with a single side slot at its distal end through which the wafers protrude as they pile up at the distal end of the tube.

In PCT/IL02/00916 (Grunberg et al., not yet published) device and method are disclosed of a plate for use in conjunction with at least another one of a plurality of other plates in a modular reconstructing and supporting assembly for reconstructing and supporting a diseased or fractured bone or within a space previously occupied by a diseased intervertebral disc of a patient. The plate is sized small enough to be suitable for separate insertion into the bone or the space, preferably through a canule, and arrangement with the other plates adjacently one on top of the other to construct scaffolding, so as to provide a supporting prosthesis. In another preferred embodiment the plate has at least two substantially opposite aspects with interlocking features designed to facilitate interlocking of adjacent plates so as to prevent or restrain their sliding off each other. Several embodiments of a deploying introduction component were described, the introduction component being basically a conduit with an inlet at one end and two substantially opposite slits at its other end, large enough to let the modular expanding element introduce itself through the introduction component and deploy in either directions through the slits, thus erecting a support structure within the bone. This introduction component is advantageous over the introducing device by Johnson et al., as the latter suggests building a wafer column in a determined direction. When inserted at the bottom of the treated vertebra (according to Johnson's described preferred approach) the base must remain supported on the floor of the vertebra in order to safely balance the load exerted on the wafers. If it is not properly supported by the vertebra's floor, the pressure of the wafer column may force it downwards, through the trabecular bone. The tube, being supported posteriorly by the cortical bone (at the point of entry to the vertebra) is exposed to substantial moment of force that tilts it forward and by that unbalances the column. The introducing device disclosed by Grunberg et al. overcomes this problem by offering two opposing slots through which the plates are constructed both up and down. It does not act as a base for the column, rather as a diverting conduit, and its actual position with respect to the column is irrelevant. Optional secure packaging of the structure was disclosed in the form of a strap that holds the structure.

### BRIEF DESCRIPTION OF THE INVENTION

There is thus provided, in accordance with a preferred embodiment of the present invention, a device for distracting and supporting two substantially opposing tissue surfaces in a patient's body, to be introduced within the tissue surfaces in a minimally invasive procedure, the device comprising:

a wrapping element;

an expandable structure insertable between the two substantially opposing support surfaces of the wrapping element, adapted to be expanded between the two substantially opposing surfaces to a predetermined dimension.

Furthermore, in accordance with a preferred embodiment of the present invention, the device of the present invention is further provided with a conduit, through which it is introduced into the patient's body.

Furthermore, in accordance with a preferred embodiment of the present invention, the device is further provided with a introduction member, the introduction member comprising a substantially linear conduit, having a proximal end through which the device is inserted and a distal end where two substantially opposite slots are provided, through which the expandable structure may protrude in directions substantially perpendicular to the conduit.

Furthermore, in accordance with a preferred embodiment of the present invention, wrapping element comprises an adjustable strap interlaced through slits that are provided on the introduction member.

Furthermore, in accordance with a preferred embodiment of the present invention, the wrapping element comprises two substantially opposing support surfaces.

Furthermore, in accordance with a preferred embodiment of the present invention, the two substantially opposing support surfaces are ragged on internal sides.

Furthermore, in accordance with a preferred embodiment of the present invention, at least one of the two substantially opposing support surfaces is provided with a protrusion for providing anchorage for the expandable structure when it is positioned between the two substantially opposing support surfaces.

Furthermore, in accordance with a preferred embodiment of the present invention, the expandable structure comprises a plurality of beams.

Furthermore, in accordance with a preferred embodiment of the present invention, the expandable structure comprises a segmented strip made of a series of jointed segments pivotally interconnected so as to present a multi-joint strip, each segment having an elongated bore provided on it through which a fastener may be interlaced, for holding the strip in a folded state of a desired height.

Furthermore, in accordance with a preferred embodiment of the present invention, there is provided a device for distracting and supporting two substantially opposing tissue surfaces in a patient's body, to be introduced within the tissue surfaces in a minimally invasive procedure, the device comprising:

a segmented strip made of a series of jointed segments pivotally interconnected so as to present a multi-joint strip, each segment having an elongated bore provided on it through which a fastener may be interlaced, for holding the strip in a folded state of a desired height.

Furthermore, in accordance with a preferred embodiment of the present invention, the expandable structure is an initially squashed deployable polyhedron structure.

Furthermore, in accordance with a preferred embodiment of the present invention, the polyhedron structure has a cross section in the form of a parallelogram.

Furthermore, in accordance with a preferred embodiment of the present invention, the device has a dual configuration.

Furthermore, in accordance with a preferred embodiment of the present invention, the expandable structure comprises two foldable straps placed on either sides of a bar.

Furthermore, in accordance with a preferred embodiment of the present invention, the expandable structure comprises a coil.

Furthermore, in accordance with a preferred embodiment of the present invention, the coil comprises a coiled strap.

Furthermore, in accordance with a preferred embodiment of the present invention, the device is further provided with a harness arrangement with two substantially parallel bars pivotally connected to an introducing conduit and coupled to an axle for the strap to be coiled on, allowing upward or downward motion of the coil with respect to the conduit.

Furthermore, in accordance with a preferred embodiment of the present invention, the coiled strap is coiled over a rotor.

Furthermore, in accordance with a preferred embodiment of the present invention, the device includes a propulsion belt for driving the strap and enhancing its coiling.

Furthermore, in accordance with a preferred embodiment of the present invention, the device is further forvided with a roller for rolling the propulsion belt.

Furthermore, in accordance with a preferred embodiment of the present invention, the belt is provided with ragged surface for enhancing friction between the belt and the coil.

Furthermore, in accordance with a preferred embodiment of the present invention, the strap is provided with a ragged surface for enhancing friction between the belt and the coil.

Furthermore, in accordance with a preferred embodiment of the present invention, the expandable structure comprises a plurality of cylindrical elements.

Furthermore, in accordance with a preferred embodiment of the present invention, the cylindrical elements are provided with cog-like surface.

Furthermore, in accordance with a preferred embodiment of the present invention, the cylindrical elements are provided with threading.

Furthermore, in accordance with a preferred embodiment of the present invention, the cylindrical elements are linked.

Furthermore, in accordance with a preferred embodiment of the present invention, the cylindrical elements are linked loosely by links that can break up when the linked cylindrical elements are pressed within the wrapping element.

Furthermore, in accordance with a preferred embodiment of the present invention, the cylindrical elements are linked by a string.

Furthermore, in accordance with a preferred embodiment of the present invention, the wrapping is incorporated with the expandable structure.

Furthermore, in accordance with a preferred embodiment of the present invention, the wrapping is incorporated with an introduction device used to introduce the device to a target location.

Furthermore, in accordance with a preferred embodiment of the present invention, the device is made form materials selected from: metal, titanium, titanium alloy, stainless steel alloys, steel 316, processed foil, hydroxyapatite, material coated with hydroxyapetite, plastics, silicon, composite materials, carbon-fiber, hardened polymeric materials, polymethylmetacrylate (PMMA), ceramic materials, coral material or a combination thereof.

Furthermore, in accordance with a preferred embodiment of the present invention, there is provided a plate for use in an assembly for distrating and supporting two opposing tissues, the assembly comprising at least one of a plurality of plates, the plate comprising a flexible structure made from a strap.

Furthermore, in accordance with a preferred embodiment of the present invention, the strap forms a structure having substantially two opposing surfaces and a portion of the strap in between the surfaces in a wavy formation.

Furthermore, in accordance with a preferred embodiment of the present invention, portions of the strap form wedge-like ends located on either sides of the substantially opposing surfaces.

Various aspects and features of the present invention will become apparent after reading the present specification and considering the accompanying figures.

### BRIEF DESCRIPTION OF THE FIGURES

In order to better understand the present invention, and appreciate its practical applications, the following Figures are provided and referenced hereinafter. It should be noted that the Figures are given as examples only and in no way limit the scope of the invention.

FIG. 1a illustrates a distraction device in accordance with a preferred embodiment of the present invention, in an initial stage of deployment.

FIG. 1b illustrates the distraction device shown in FIG. 1a in an intermediate stage of deployment.

FIG. 1c illustrates the distraction device shown in FIG. 1a in a progressive stage of deployment.

FIG. 2a illustrates a wrapped distraction device in accordance with a preferred embodiment of the present invention.

FIG. 2b illustrating a wrapped distraction device in accordance with a preferred embodiment of the present invention incorporated with a introduction device.

FIG. 3a illustrates an adjustable support structure to be used in conduction with a distraction device in accordance with the present invention, in an initial deployed state.

FIG. 3b illustrates the support structure of FIG. 3a in an intermediate state.

FIG. 3c illustrates the support structure of FIG. 3a in a final state.

FIG. 4a illustrates another preferred embodiment of a distraction device in accordance with the present invention, in its initial squashed state.

FIG. 4b illustrates a dual configuration of the device shown in FIG. 4a.

FIG. 4c illustrates the dual configuration of FIG. 4b in a deployed state.

FIG. 4d illustrates the dual configuration of FIG. 4b in a deployed state, in conjunction with a introduction device.

FIG. 5a illustrates another preferred embodiment of a distraction device in accordance with the present invention, in an initial cramped state.

FIG. 5b illustrates the device shown in FIG. 5a, in a deployed state.

FIG. 6a illustrates yet another preferred embodiment of a distraction device in accordance with the present invention, incorporating two support surfaces and a coil (the coil not yet deployed).

FIG. 6b illustrates the device shown in FIG. 6a, with the strip to be coiled up within the support surfaces engaged to the coiling rotor.

FIG. 6c illustrates the device shown in FIG. 6a in a fully deployed state.

FIG. 6d illustrates the use of two devices as shown in FIG. 6b, in a parallel manner.

FIG. 7a illustrates another embodiment of a distraction device in accordance with the present invention, with a propulsion belt.

FIG. 7b shows a slightly modified version of the device shown in FIG. 7a.

FIG. 7c illustrates an embodiment of a propulsion belt in accordance with the present invention.

FIG. 7d illustrates another embodiment of a propulsion belt in accordance with the present invention.

FIG. 7e illustrates yet another embodiment of a propulsion belt in accordance with the present invention.

FIG. 7f illustrates another modified version of the device shown in FIG. 7a.

FIG. 7g illustrates the device shown in FIG. 7a with a roller for actuating the propulsion belt and rolling the coil at the target location.

FIG. 8a illustrates another preferred embodiment of the distraction device of the present invention, incorporating a plurality of rigid cylinders within a wrapping.

FIG. 8b illustrates another preferred embodiment of the distraction device of the present invention, incorporating a plurality of rigid cylinders within a wrapping, in conjunction with a introduction device.

FIG. 9 illustrates a modified version of the device of FIG. 8, with the cylinders provided with a ragged cog-like surface.

FIG. 10 illustrates an introduction device with an adjustable wrapper and a plurality of cylinders serving as intermediary support structure.

FIG. 11 illustrates another modified version of the device of FIG. 8, with a plurality of cylindrical elements provided with threadings.

FIG. 12a illustrates a chain of elements suitable for use as filling for an intermediary support structure inside a wrapping in accordance with the present invention.

FIG. 12b illustrates another view of the chain of elements portrayed in FIG. 12a.

FIG. 13a illustrates another shape of a chain of elements suitable for use as filling for an intermediary support structure inside a wrapping in accordance with the present invention.

FIG. 13b illustrates another view of the chain of elements portrayed in FIG. 13a.

FIG. 13c illustrates yet another embodiment of a chain of elements suitable for use as filling for an intermediary support structure inside a wrapping in accordance with the present invention.

FIG. 14 illustrates an embodiment of a shock absorbing support element.

FIG. 15a illustrates another embodiment of a shock absorbing support element.

FIG. 15b illustrates the shock absorbing support element of FIG. 15a in a compressed state.

FIG. 16 illustrates yet another embodiment of a shock absorbing support element.

FIG. 17 illustrates a holder for accurate deployment of the distraction device in accordance with the present invention.

FIG. 18 illustrates a fixator for fixing in position two introduction devices in accordance with the present invention in deployment.

### DETAILED DESCRIPTION OF THE INVENTION AND FIGURES

The present invention deals with a distraction device for distracting and supporting two opposite tissue surfaces within a body.

The device according to the present invention is a modular and/or gradual tissue distractor. The distraction is achieved by a structure that expands perpendicular to and in between the surfaces to be distracted apart. Fundamentally, the expandable structure has a large freedom of movement to actively seek the position of least resistance. When expanding, this feature guarantees the equal administration of the distraction force to the two facing tissue surfaces.

The distraction device fundamentally consists of: a conduit that optionally acts as a barrier and divertor (to the movement of the expandable element other than perpendicular to the conduit), an expandable element, and a wrapping element.

The wrapping element may be a wrapping strap, string, bag or any other wrapper. It may also include two or more parts, such as two opposing support surfaces that the expandable element is held in between them.

The expandable element is fed or grows into the wrapping element that wraps, compacts, and ties it to the conduit. Due to the nature of the wrapping element, it controls the level of and to some degree the shape of the expandable element. This wrapping element is the one that comes in contact with the tissue surfaces and diverts them form one another. Optionally, two intermediate substantially opposing surfaces are added between the wrapping element and the tissue surfaces being districted to better regulate the device's “foot print” on the distracted tissue surfaces. The foot-print of the device relates to the area being forced or pressed apart, the shape and texture of the surface and roughness of these surfaces. Another option is that at the end of expanding, the conduit may separate from the expandable unit, at the end of the medical procedure.

The expandable element can be for example:

A structure that is forced to erect when surrounded by the wrapping element (a quardrihedron such as parallelogram, trapezoid, or a beam structure, and other similar shapes)

A filler consisting of a plurality of separated elements that are forced into the wrapping element. Once in the wrapping element they randomly fill and occupy the space within. There is a variety of solutions (cylindrical rollers, elliptic profile segments, tiles/plates/wafers) suitable for use as fillers. For the purpose of the present invention the term “cylindrical” is used to refer to any element having a predetermined profile along substantially constant length, and not only elements with a circular profile.

A filler that is a continuous element forced to coil into the wrapping element (e.g. coiled strip, coiled chain elements, coiled toothed strip etc.).

Alternatively, the filler may be a continuous element of loosely chained components (e.g. stringed cylindrical rollers, stringed elliptic profile segments, etc,). The filler is chained when fed through the conduit, to allow continues action and easy feeding. However, when compacted into the containing element, the loosely chained elements readily break up into segments and randomly organize.

The expandable element may be separated from the wrapping element, or coupled to it, or it may be coupled to an introducing conduit or any other introduction device.

A main aspect of the present invention is the provision of a distraction and support device comprising a wrapping envelope that is capable of distracting two tissue surfaces apart and keep them at a predetermined distance.

The distracting surfaces of the wrapping element are designed to distract and support two opposite tissue surfaces within a body, such as the opposing cortex of a compressed vertebra, with the intermediary structure bearing the load or forces acting upon the support surfaces of the wrapping element, or maintaining the distance between the two opposite surfaces.

The nature of the intermediary support structure may vary. The intermediary support structure may, for example, be a deployable device or comprise a plurality of objects that are disposed between the support surfaces and convey or distract them apart, holding the load exerted on the overall structure.

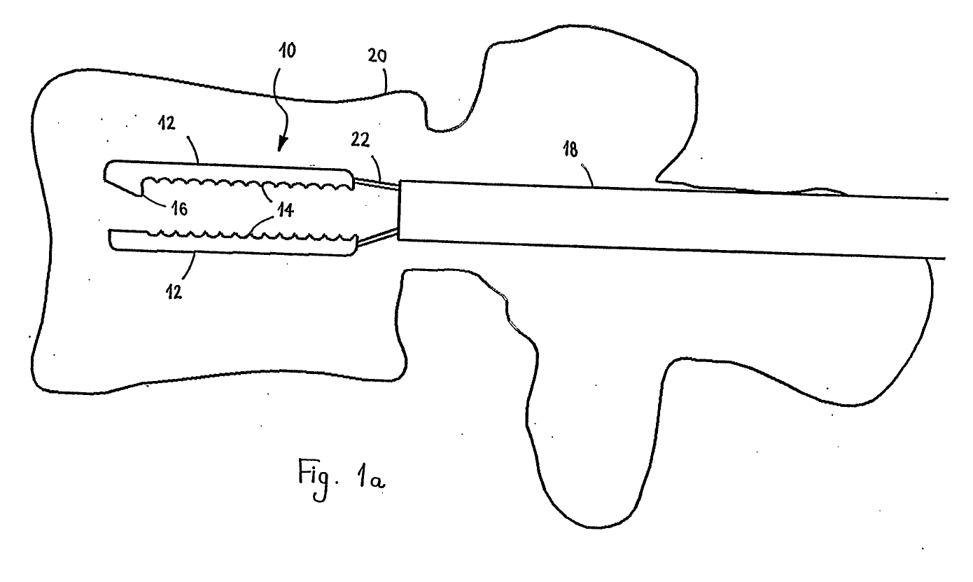

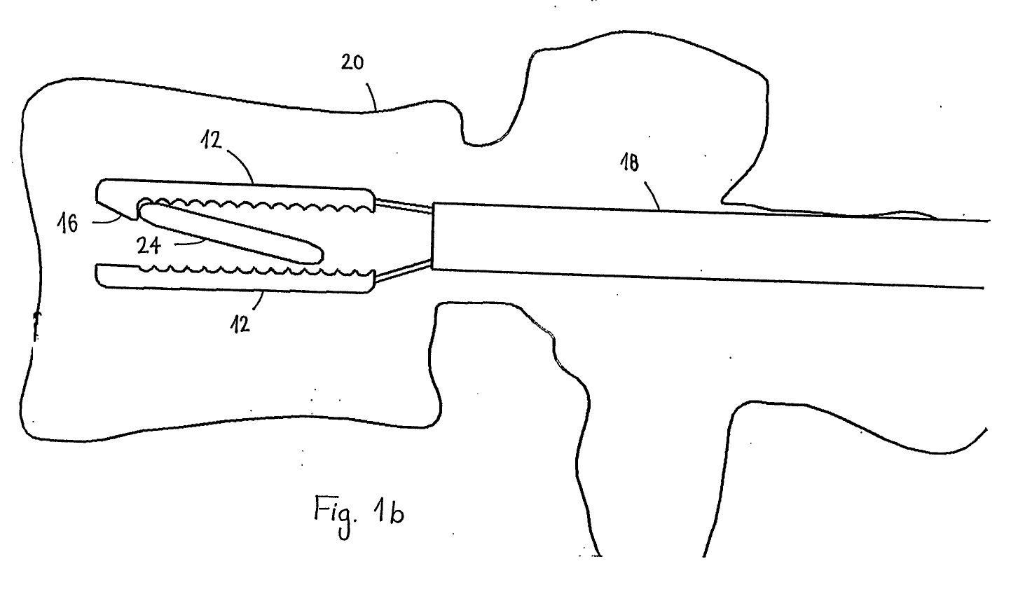

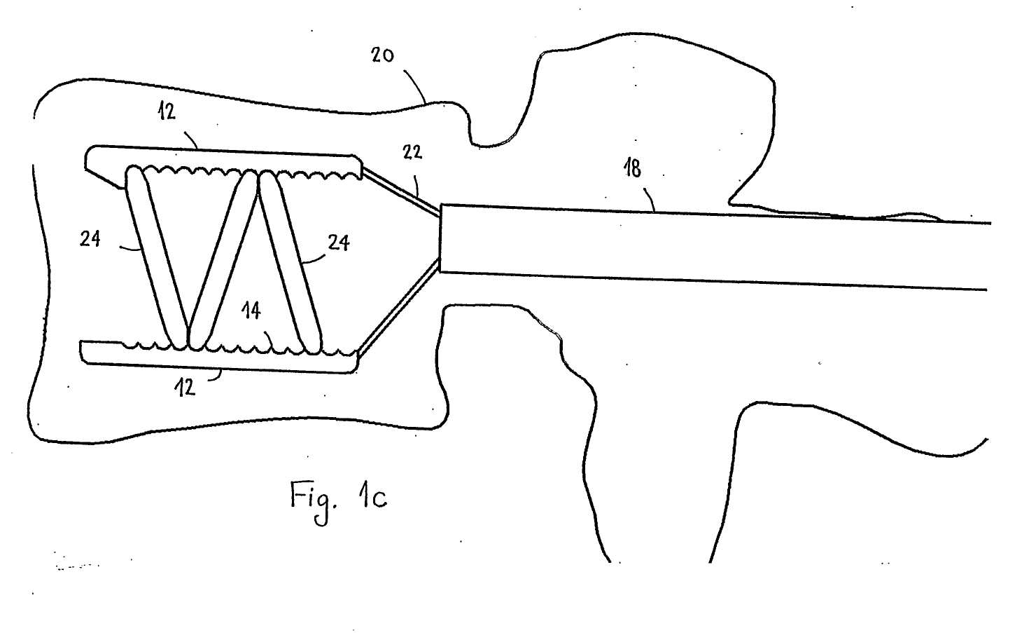

Reference is now made to FIG. 1a illustrating a distraction device 10 in accordance with a preferred embodiment of the present invention, in an initial stage of deployment, used within a vertebra.

Two support surfaces 12 are introduced into a vertebra 20 through a bore drilled in the vertebra (for example in the pedicle), using an introduction component in the form of a conduit 18, through which all parts of the distraction device are introduced into the desired target location. This is a minimally invasive procedure, involving only a small incision in the patient's skin and insertion of small-diameter introduction tools, such as a drill and conduit 18, through that incision towards the target location. Once on location within the body, be it inside a vertebra, or inside other body cavity or between two opposite tissue surfaces, the distraction device is constructed and erected to its desired height.

The support surfaces 12 are initially kept within the conduit 18 and are advanced out of the conduit's distal end using an advancement tool (not shown in the figure), and are coupled to the conduit by coupling means such as strips 22 (or cables or arms etc.), each strip coupled to a support surface. The strips are manipulated from the proximal end of the conduit (not shown in the figure), which is further from the target location, and most conveniently outside the patent's body.—

The support surface 12 may preferably have a protrusion 16 at its further end, designed to provide anchorage for the intermediary structure, which is to be inserted between the support surfaces 12 and deployed.

Reference is now made to FIGS. 1b and 1c illustrating the distraction device shown in FIG. 1a in an intermediate stage of deployment and in a progressive stage of deployment.

A beam 24 is introduced between the two support surfaces with the help of an introducing tool such as a rod or similar such device (not shown in the figure). The beam is blocked by protrusion 16 and as it is pushed further from the conduits direction by the introducing tool it is raised up to an upright position pushing the support surfaces apart. More beams 24 are consecutively introduced between the support surfaces until an intermediary structure is formed there, supporting both surfaces and holding them separated, carrying the load exerted by the distracted surfaces. The inner side of each support surface may be ragged to provide anchorage for the tips of the beams preventing slipping of the beams and consequent collapse of the whole device.

The introduction device 18 may then be altogether removed, or remain in position, allowing the bone or other surrounding tissue to grow over it. For the latter to be possible, the introduction device may be formed of a detachable parts the final part being the part shown in the drawings, so that the remaining parts of the introduction device are disconnected from the final part and removed.

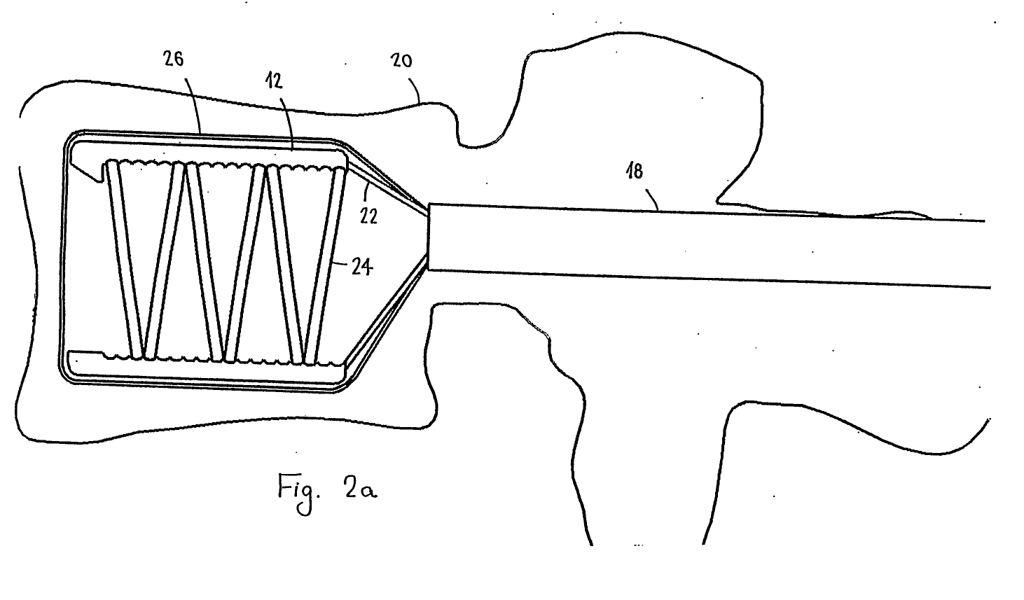

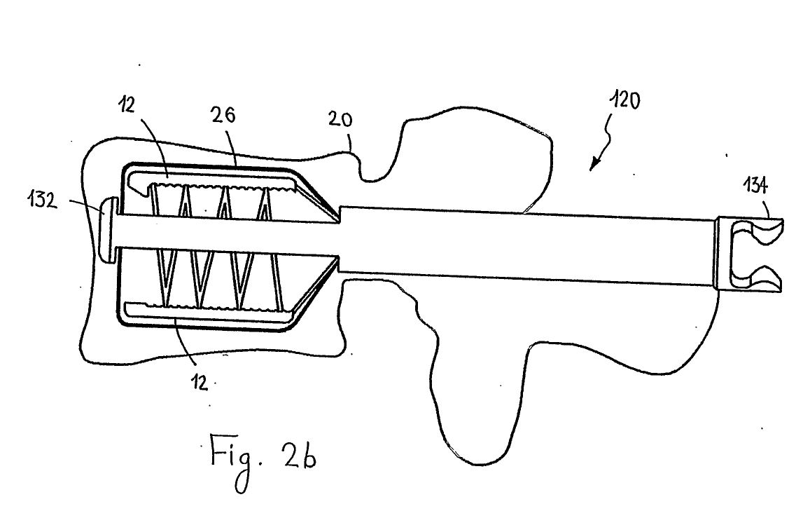

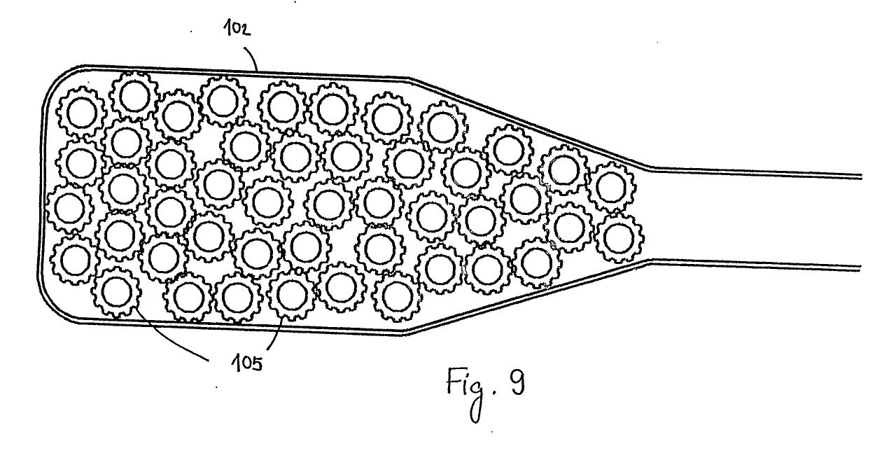

FIG. 2a illustrates a distraction device incorporating a wrapper in accordance with a preferred embodiment of the present invention. In this configuration of the present invention, a wrapping strap 26 is provided to secure the entire expanding element in its final configuration. The strap may optionally be incorporated with the introduction device (see for example FIG. 9 and FIG. 10).

FIG. 2b illustrating a distraction device incorporating a wrapper in accordance with a preferred embodiment of the present invention incorporated with an introduction component (further explanation on the introduction component is provided hereinafter).

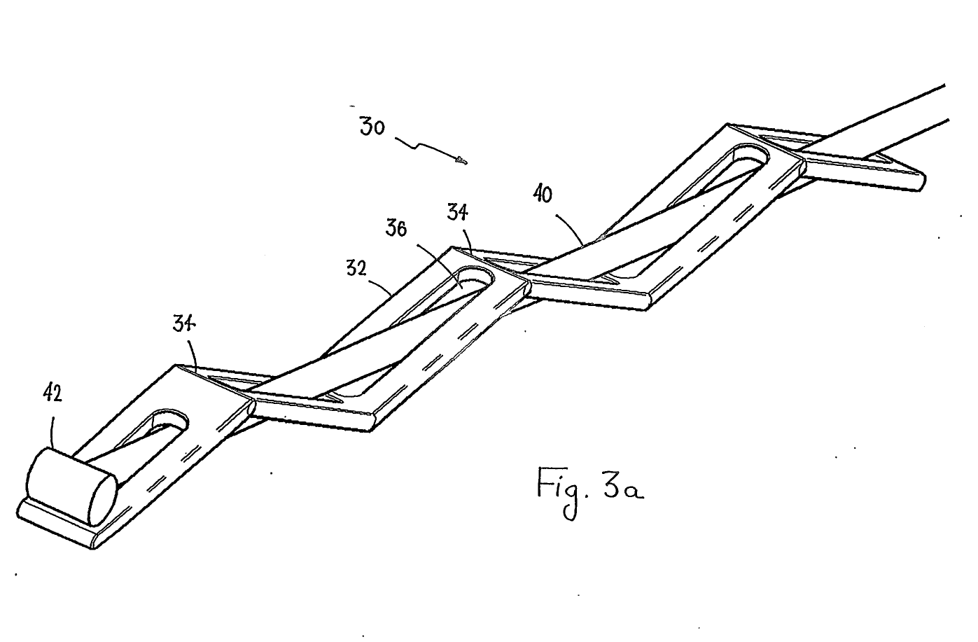

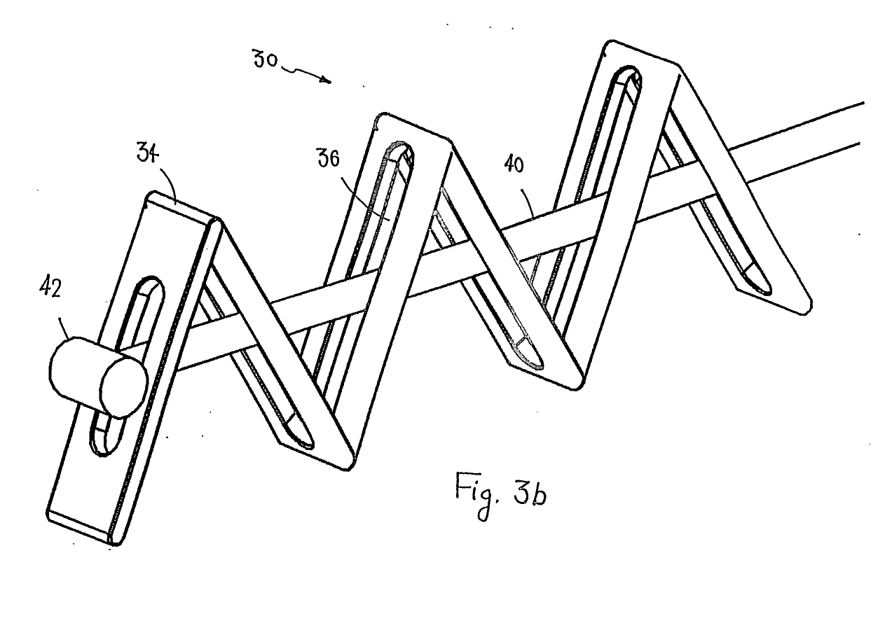

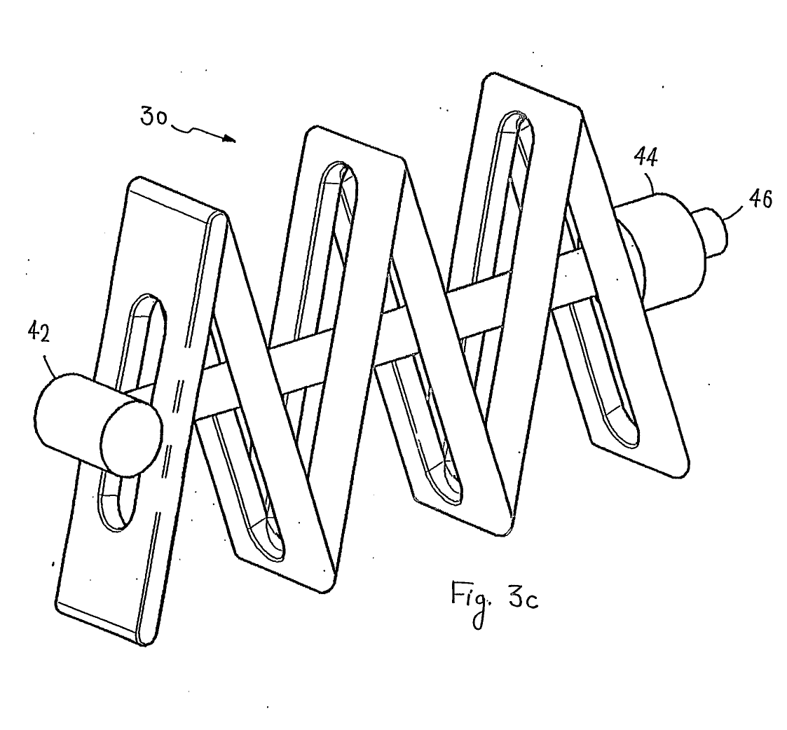

FIG. 3a illustrates an adjustable intermediary support structure to be used in conjunction with a distraction device in accordance with the present invention, in an initial deployed state. The adjustable structure comprises a strip 30 of a rigid material (such as titanium, steel 316, or other biocompatible material) comprising a series of boards 32 pivotally (34) interconnected so as to present a multi-joint strip. Each board 32 has an elongated bore 36 provided on it through which a rod 40 is interlaced. At a distal end of the rod a stopper 42 is provided, wider than the bore, preventing the last board from falling off the rod. The pivots may be mechanical, such as hinges, or just thinner areas of material, which are therefore more susceptible to bending. It is noted that it is anticipated that the intermediary support structure be installed in a one-time manner, and therefore a mechanical hinge may be both too expensive and too delicate for the job. However a mechanical hinge, and in fact any other solution that allows the strip to be bent at the joints is acceptable and covered by the scope of the present invention.

FIG. 3b illustrates the support structure of FIG. 3a in an intermediate state. FIG. 3c illustrates the support structure of FIG. 3a in a final state. Once on the target location, the proximal end of the last board may be pushed forward, causing the entire strip to be compacted and hence bend at the pivots, rising in height as it does so. When the desired height is reached a bolt 44 (or any other stopper) is fixed on the rod to prevent the strip from deploying backwards, and the remaining rod is removed, or cut off.

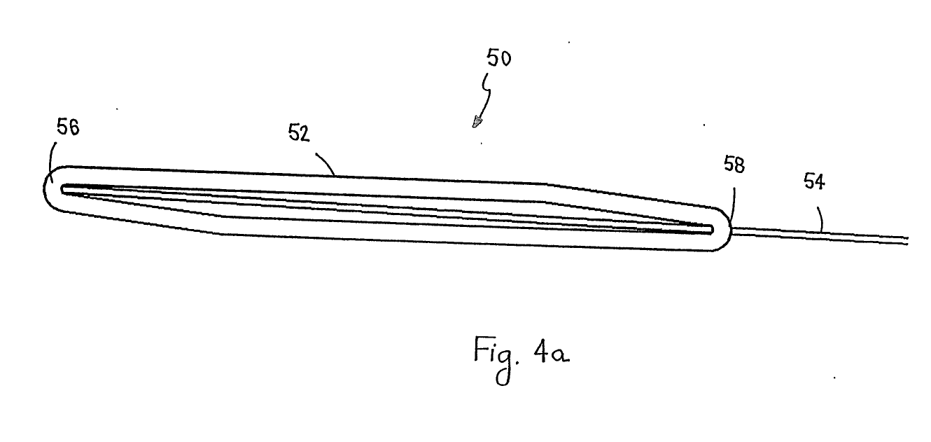

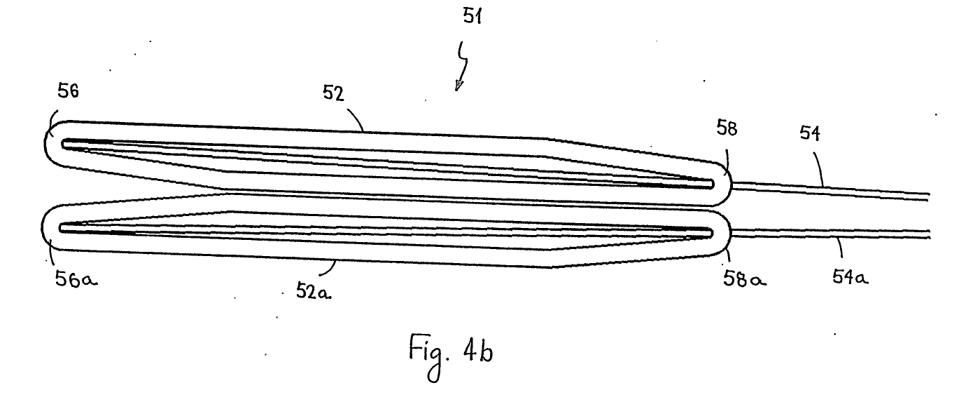

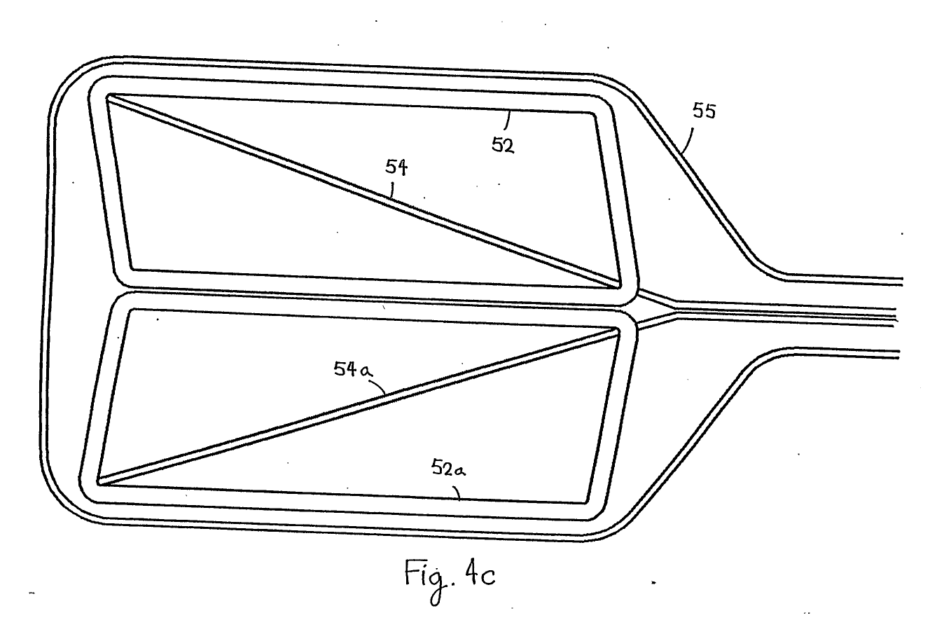

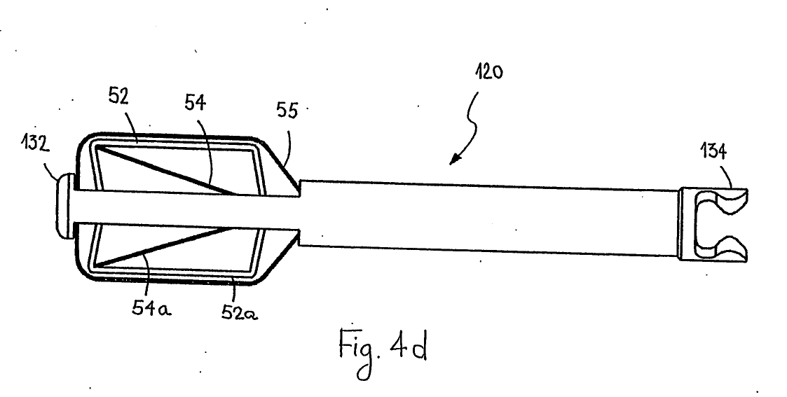

FIG. 4a illustrates another preferred embodiment of a distraction device in accordance with the present invention, in its initial squashed state. A squashed structure 50, whose cross section is a polyhedron structure having a cross section in the form of a parallelogram 52 (or a trapezoid), with a diagonal element in the form of a strip 54, coupled to the far end of the parallelogram 56 and passing through the opposite end 58. When the strip is pulled, with end 58 secured in position, the entire structure rises and deploys. The support surfaces that distract and support the two opposite tissue surfaces are its two parallel sides (top and bottom). Alternatively two additional support surfaces may be provided, with the device shown in FIG. 4a positioned in between them.

FIG. 4b illustrates a dual configuration 51 of the device shown in FIG. 4a. Here a second parallelogram structure 52a is provided side by side to the first one (52), with diagonal strip 54a coupled to far end 56a and passing through opposite end 58a. The deployment of this configuration involves pulling both strips—54, 54a, while holding ends 58 and 58a in position.

FIG. 4c illustrates the dual configuration of FIG. 4b in a deployed state. Optional wrapping strap 55 may be provided to hold the deployed structure in position. The narrowing of the strap residue on the right of the figure suggests it is passed through an introduction device in the form of a conduit (see device 18 in previous figures).

FIG. 4d illustrates the dual configuration of FIG. 4b in a deployed state, in conjunction with an introduction component.

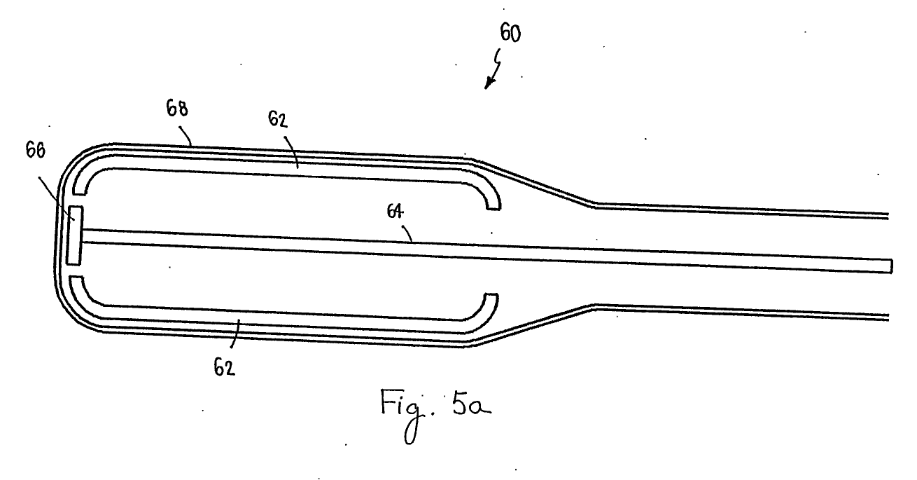

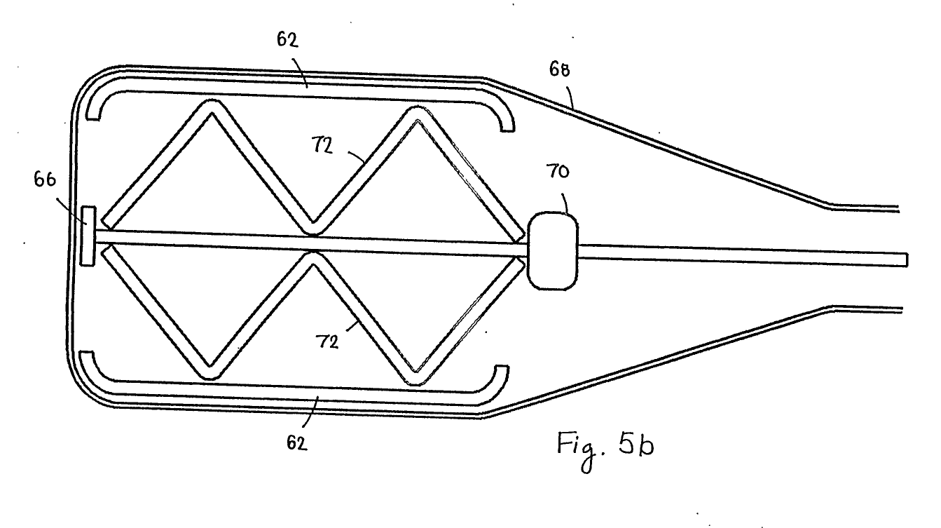

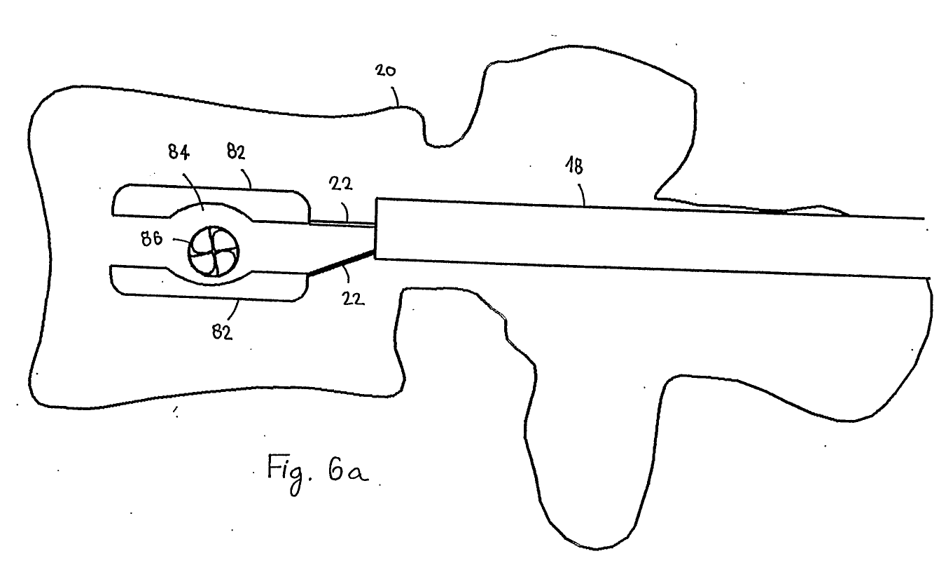

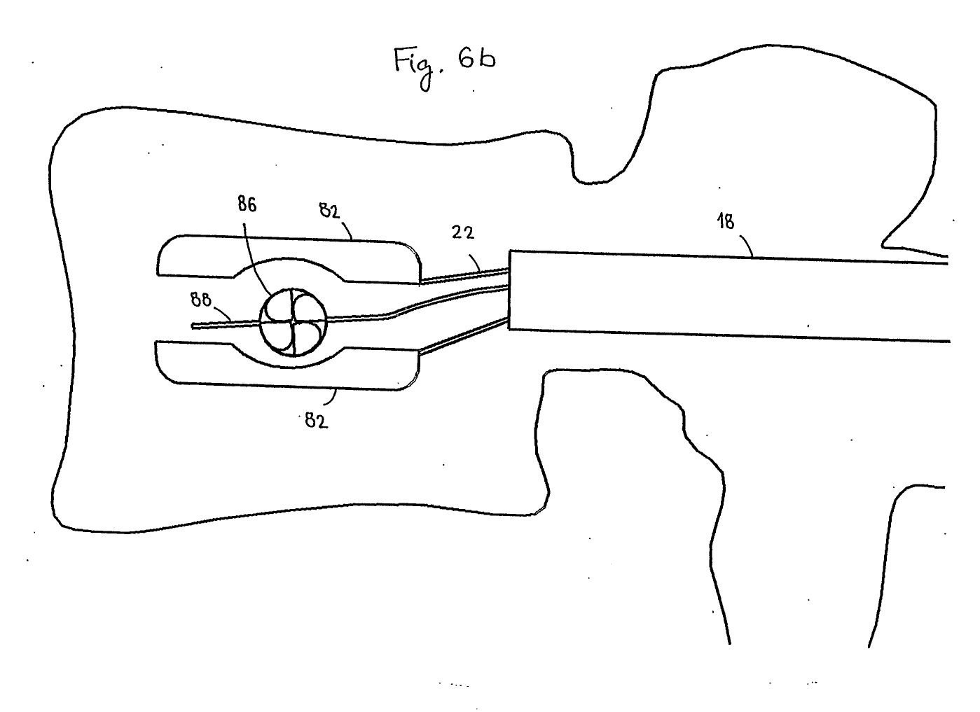

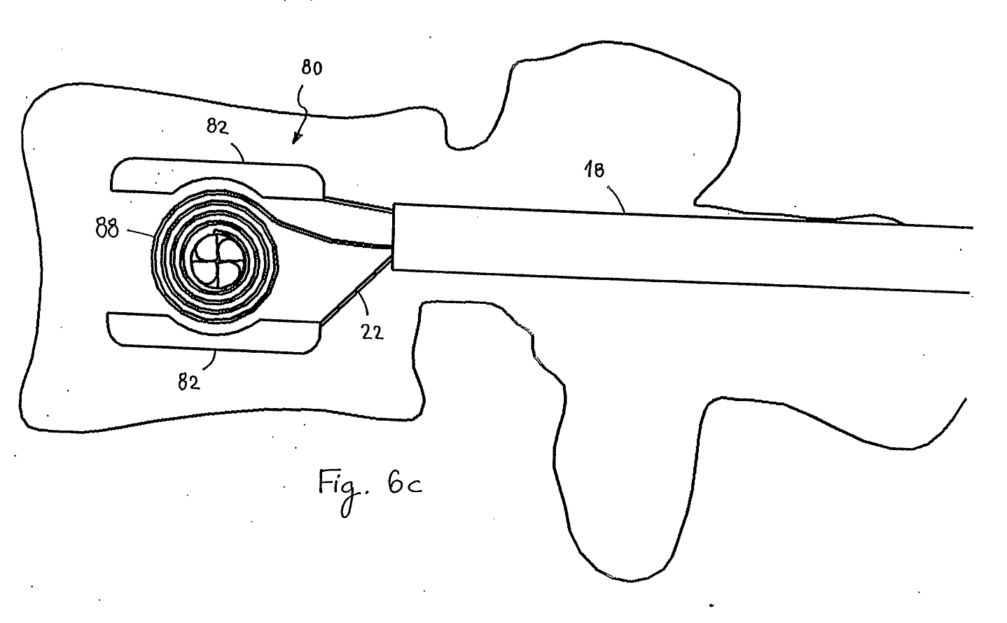

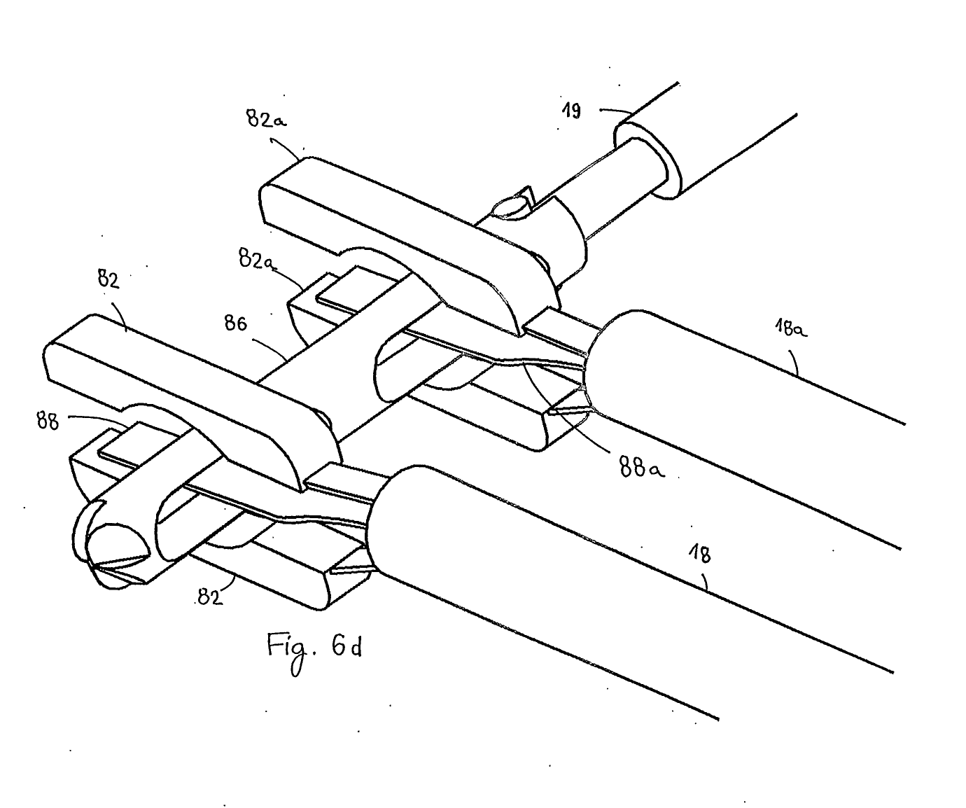

FIG. 5a illustrates another preferred embodiment of a distraction device 60 in accordance with the present invention, in an initial cramped state. FIG. 5b illustrates the device shown in FIG. 5a, in a deployed state. The device comprises a wrapping strap 68 holding two substantially opposite support surfaces 62 located on either sides of a central bar 64 provided with a stopper 66 at its distal end. A foldable multi-joint strip 72 is provided on either sides of the bar, pressed on the proximal end by an adjustable stopper 70. The foldable multi-joint strip may be of the same sort as the strip shown in FIGS. 3a-3c. FIG. 6a illustrates yet another preferred embodiment of a distraction device in accordance with the present invention, incorporating two support surfaces and a coil (the coil not yet deployed). Similarly to the device shown in FIGS. 1a-1c, the support surfaces could initially be introduced through the introducing conduit 18 to the target location (for example within a vertebral body 20), and are advanced out of the conduit by strips 22, to which they are optionally attached to. An optional circular recess 84 is provided at the internal sides of the support surfaces. A rotor 86 is introduced in between the two support surfaces 82. The rotor may be integral to the device or separately introduced substantially perpendicular to the conduit 18 (see explanation of FIG. 6d). FIG. 6b illustrates the device shown in FIG. 6a, with a strip 88 to be coiled up within the support surfaces engaged to the rotor. FIG. 6c illustrates the device shown in FIG. 6a in a fully deployed state. The rotor is rotated and as it rotates the strip is coiled around the rotor. The mere coils are added the larger the coiled structure becomes, distracting and supporting the two opposite support surfaces 82. FIG. 6d illustrates the use of two devices as shown in FIG. 6b, in a parallel manner. Here two substantially parallel distraction devices are used, comprising two opposite support surfaces 28, 82a, strip to be coiled 88, 88a, and a joint rotor 86 which is introduced substantially perpendicular to the direction of the introduction conduits 18, 18a. The two opposite support surfaces are not essential here and may be omitted.

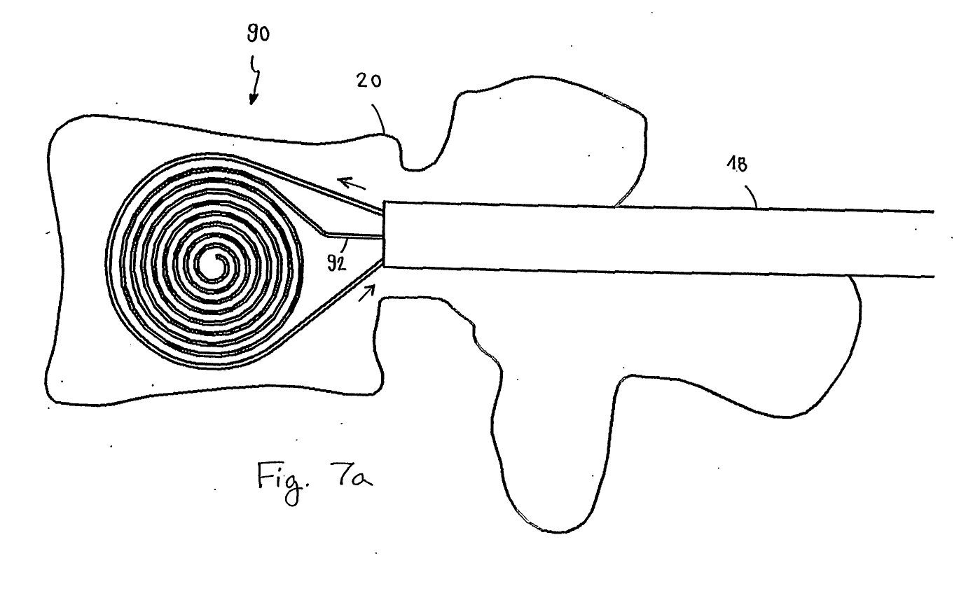

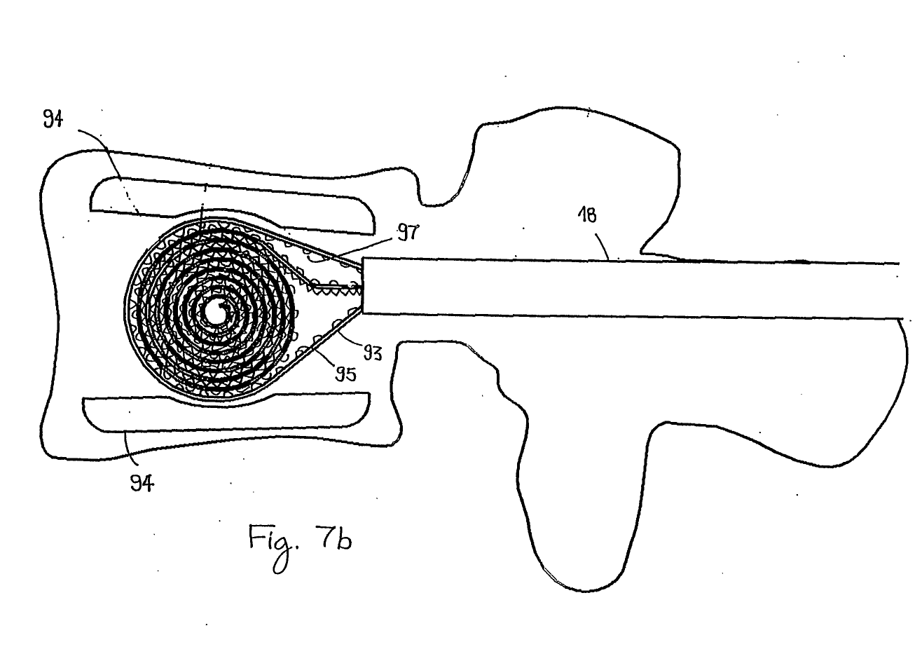

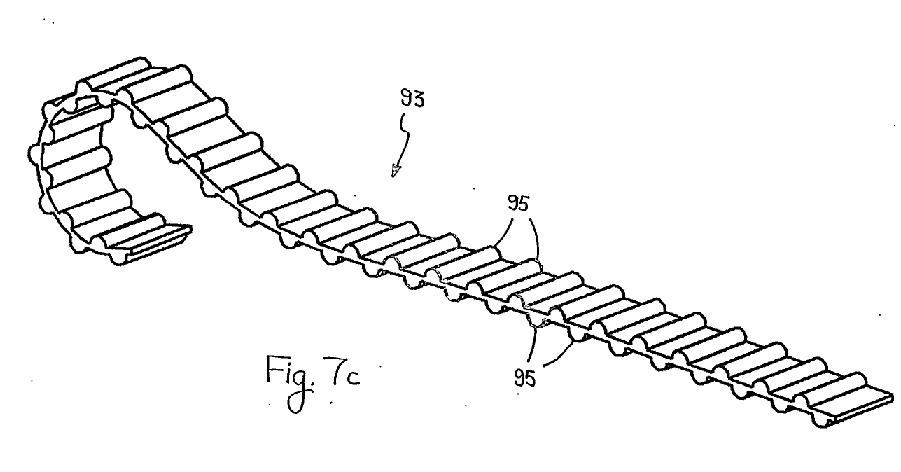

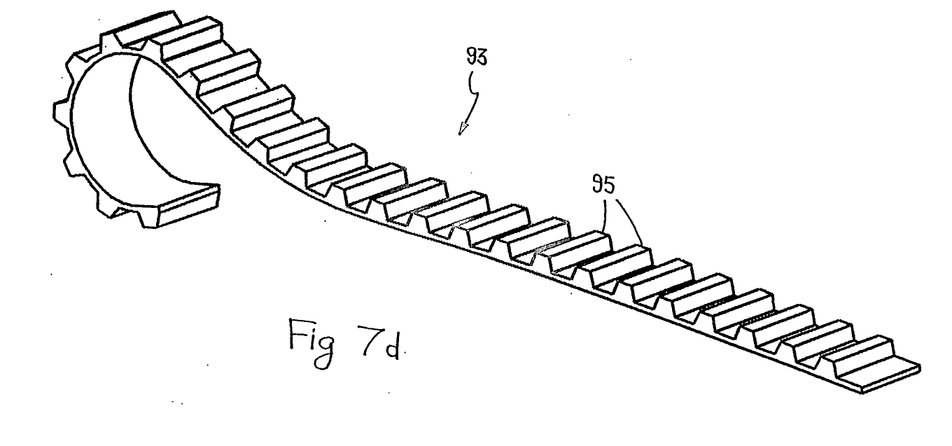

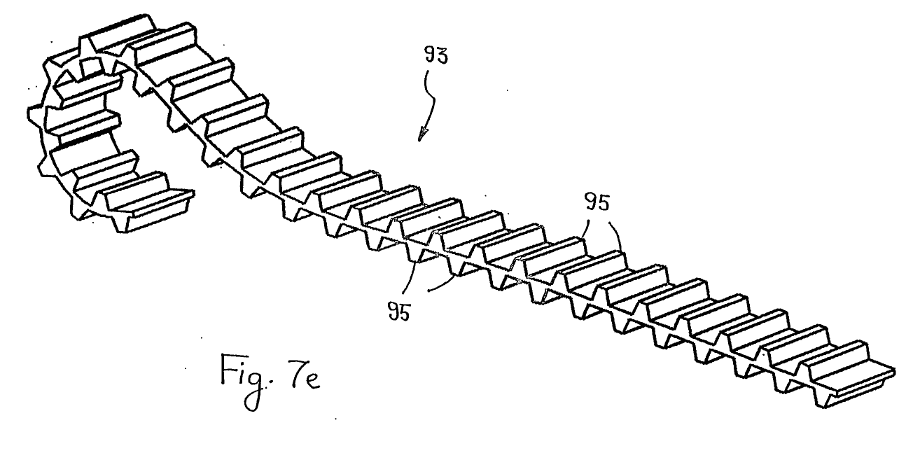

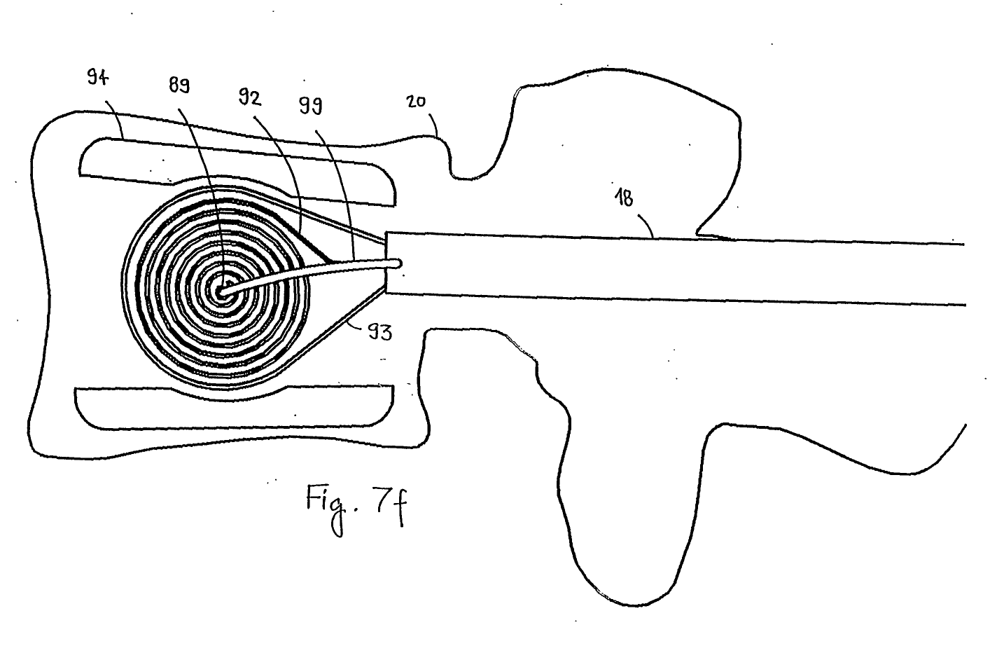

FIG. 7a illustrates another embodiment of an intermediary support structure 90 in accordance with the present invention, with a propulsion belt. The structure at hand is, like in the device shown in FIGS. 6a-6d, a coiled strip. However the strip 92 is coiled using a propulsion belt 93, which is operated from the proximal end (not shown in this figure) of the introduction conduit 18. The propulsion belt drives the strip and forces it to coil, exerting rotational force on the already coiled portion of the strip.

FIG. 7b shows a slightly modified version of the device shown in FIG. 7a. Here the strip and/or the belt have ragged or toothed surfaces 97, 95 so as to increase the friction exerted between the belt and the strip and enhance the grip between the belt and the strip.

FIG. 7c illustrates an embodiment of a propulsion belt 93 in accordance with the present invention. The belt is provided with periodical lateral protrusions in the form of bumps 95 serving to enhance friction. These bumps may be provided on both sides of the belt (as shown in FIG. 7c or on either of the sides, as shown in FIG. 7d). FIGS. 7d and 7e illustrates other embodiments of a propulsion belt in accordance with the present invention with bumps of different shapes (here having trapezoidal cross section).

FIG. 7f illustrates another modified version of the device shown in FIG. 7a. A harness arrangement in the form of two substantially parallel bars 99 pivotally connected to the conduit and provided with an adjoining axle 89 on which the strip may be coiled is provided. The strip may be pushed forward from its distal portion by a tool that forces it to move forward and be coiled on the axle. The axle is free to move up or down thus allowing the device to find its natural position when deployed. At the same time this arrangement cooperates with the pushed-forward strip providing counter force against the push of the strip and thus enhances the ability of the strip to coil on the axle.

FIG. 7g illustrates the device shown in FIG. 7a with a roller 300 for actuating the propulsion belt and rolling the coil at the target location. The roller is provided with a drum 306 on which the strap 92 is initially coiled on. The propulsion belt 93 is rolled over wheel 302 at the proximal end of the conduit 18 and over another coiled portion of the strap on wheel 302b fixed on arm 91 coupled to the conduit on its distal end. As arm 304 is rotated to roll wheel 302a, which moves propulsion belt 95. The belt advances the strap until all of it is coiled over wheel 302b.

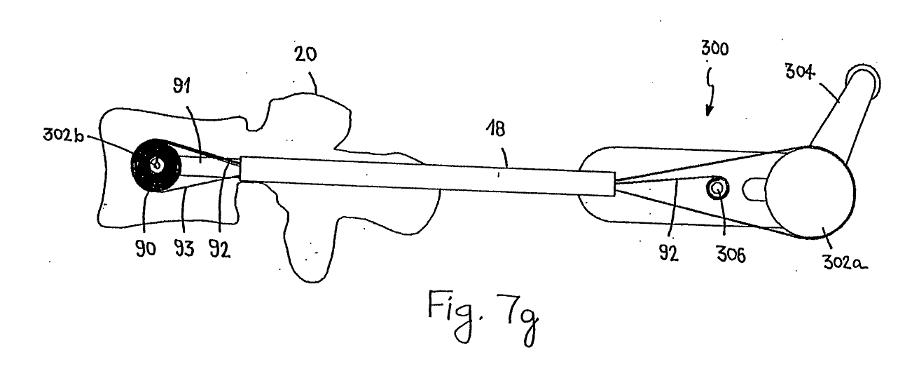

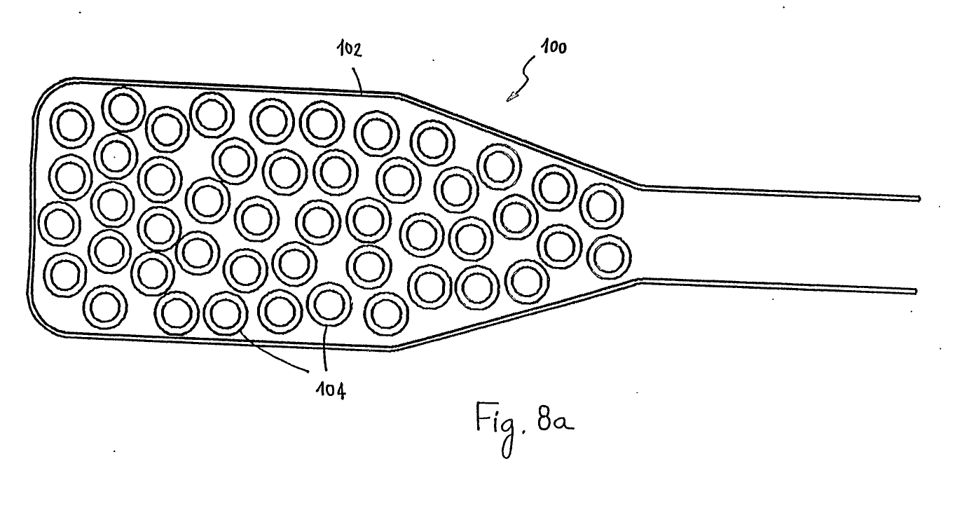

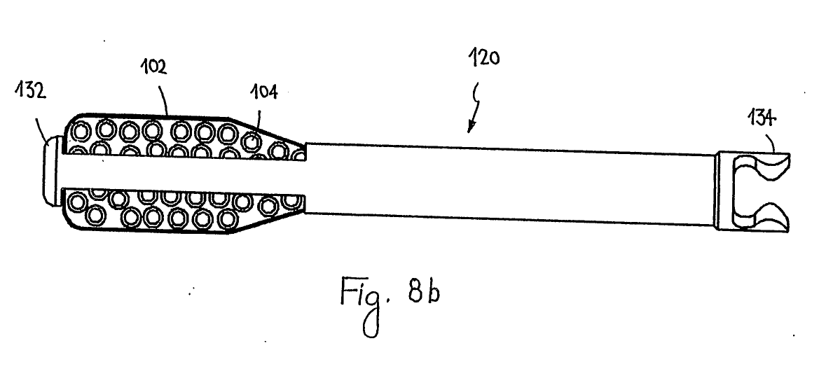

FIG. 8a illustrates another preferred embodiment of the distraction device of the present invention, incorporating a plurality of rigid cylinders within a wrapping. This is a different approach to the intermediary support structure altogether. Here the device 100 is made up of a plurality of cylinders 104 packed inside a wrapping 102, the wrapping covering the cylinders from all sides in the form of a bag, or as a wrapping strip, across the cylinders. In an alternative embodiment the entire package is introduced using an introducing device which is in fact a tube 110 with two lateral substantially opposite slits at its distal end, allowing a deploying structure introduced through the tube to emerge from the slits. When the cylinders fill the wrapping and are compressed the wrapping expands through the slits and extends beyond the boundaries of the tube.

FIG. 8b illustrates a modified version of FIG. 8, in accordance with a preferred embodiment of the distraction device 100 of the present invention, incorporating a plurality of rigid cylinders 104 within a wrapping 102, in conjunction with a introduction device.

FIG. 8b illustrates another preferred embodiment of the distraction device of the present invention, incorporating a plurality of rigid cylinders within a wrapping, in conjunction with a introduction device 120.

FIG. 9 illustrates a modified version of the device of FIG. 8, with the cylinders 105 provided with a ragged cog-like surface. Here the cylinders are provided with rough surfaces for better stability, as they are less prone to rotational movement that may introduction to collapse of the structure during the building states of the structure.

FIG. 10 illustrates an introducing component with an adjustable wrapper and a plurality of cylinders serving as intermediary support structure. The introduction component 120 is provided with two channels (slots) 126 through which the end of the wrapping strap 122 is inserted, having a bulged stopper 130 at the end to prevent it from slipping out of the channel. As more and more cylinders 136 (or other intermediary support structure) are introduced the intermediary support structure begins to bulge outside the slots 128 with the wrapping strap 122 protruding further, the distal end of the conduit (the introduction device) being blocked by a stopper 132. As a result, the bulged ends 130 slide along the channel 126, allowing the strap to be extended to the required dimension outside the slots 128. The proximal end of the introduction device 120 is designed to be engaged to an introducing device, such as a rod, and therefore is provided with engagement means in the form of connector 134 having dents and protrusions in a predetermined arrangement and matching to corresponding connector to be provided on the holder which is used to guide the device to its target location.

FIG. 11 illustrates another modified version of the device of FIG. 8, with a plurality of cylindrical elements provided with threading 107. The threading assist in preventing lateral movement or escape of elements from the structure.

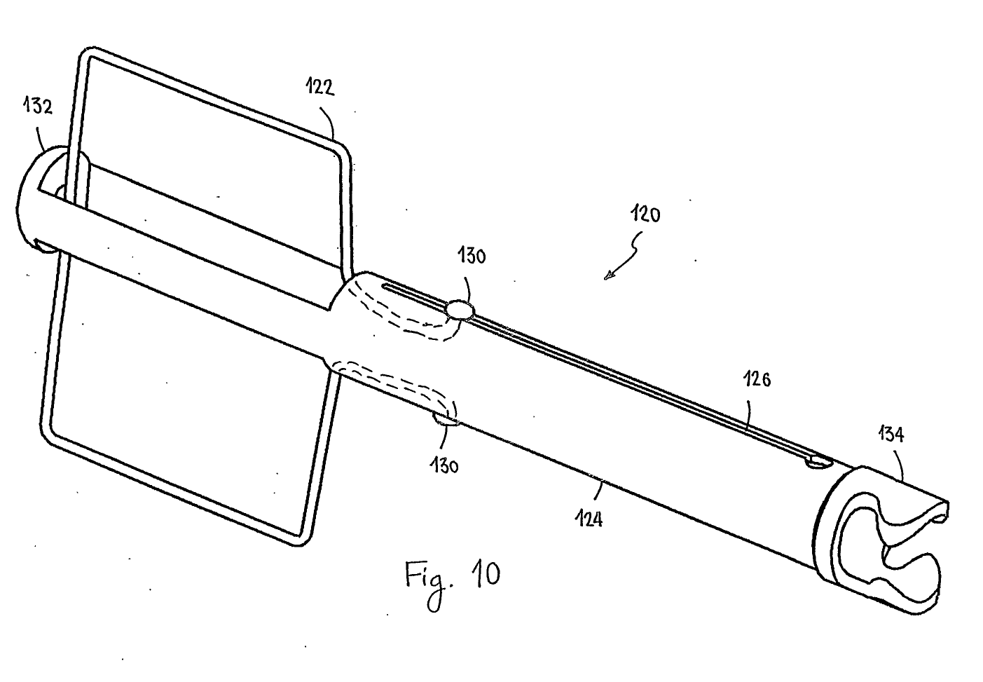

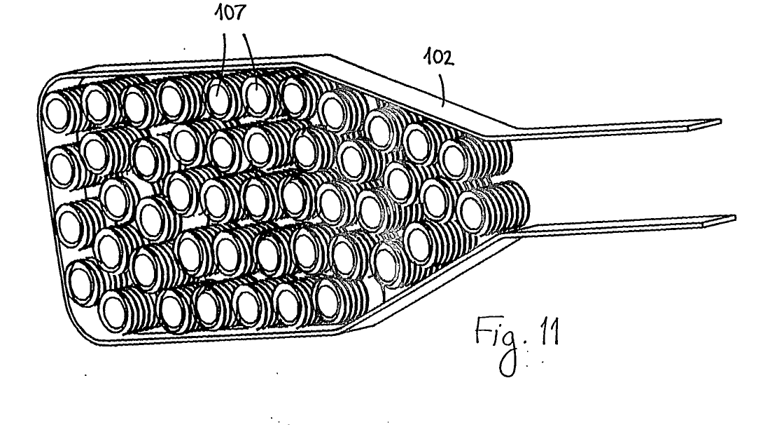

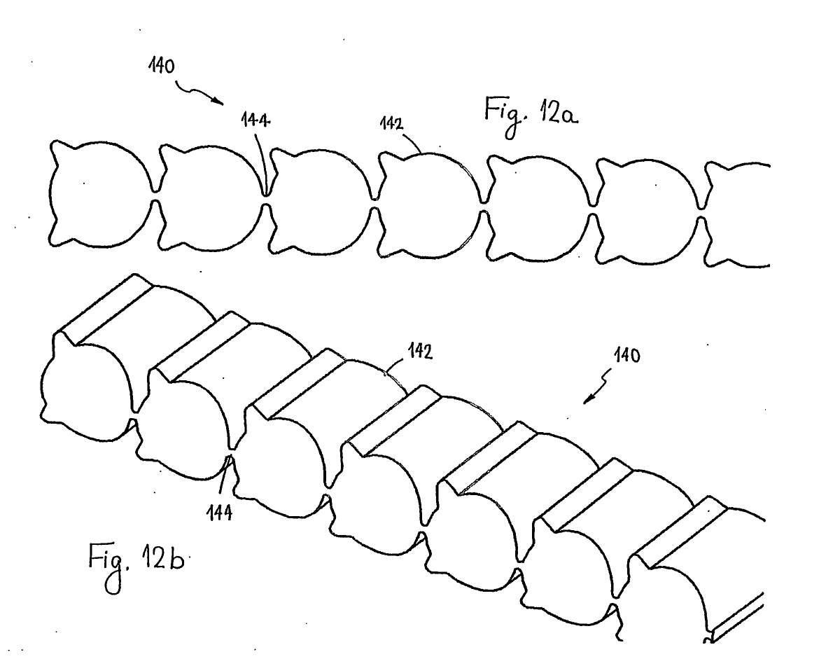

FIG. 12a illustrates linked elements suitable for use as filling for an intermediary support structure inside a wrapping in accordance with the present invention. The elements connected by contacts 144, which are preferably easily breakable and may be snapped upon the application of force on the contact, such as pressure applied in a compressed wrapping. The chain 140 of elements 142 may be continuous or comprised of only several elements and is introduced into the space bound by the wrapping through a conduit or similar introduction device. As it is accumulated within the wrapping contacts 144 between the elements may break separating some or all of the elements, thus allowing greater dispersion of the elements within the wrapping. The contacts may even comprise gluing of two elements at adjoining sides. FIG. 12b illustrates another view of the chain of elements portrayed in FIG. 12a.

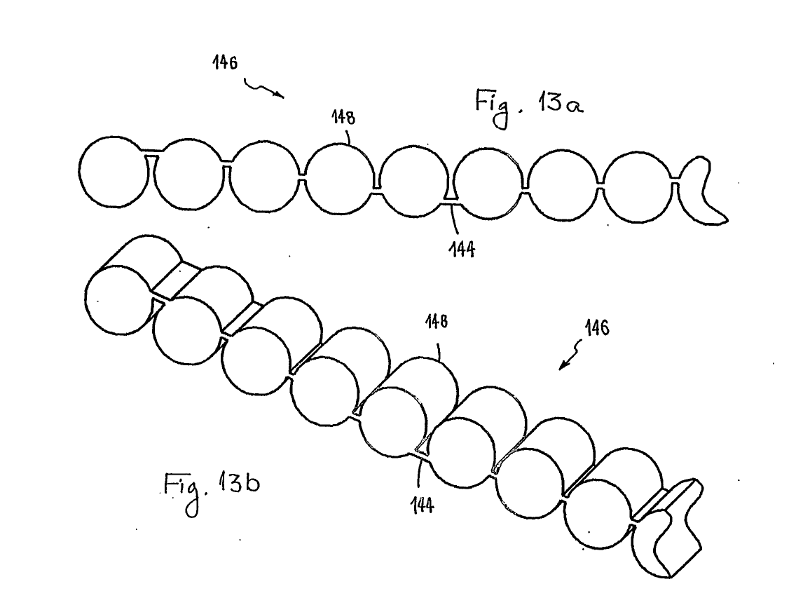

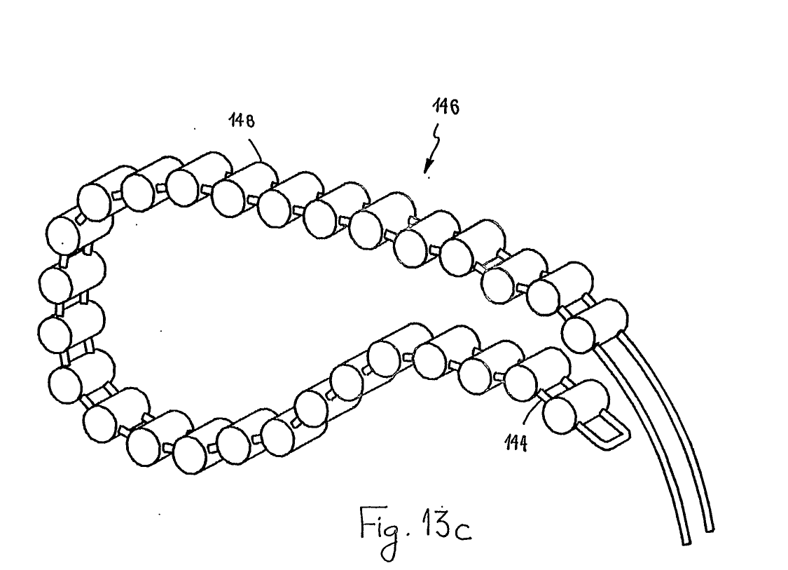

FIG. 13a illustrates another shape of a chain 146 of elements 148 suitable for use as filling for an intermediary support structure inside a wrapping in accordance with the present invention. FIG. 13b illustrates another view of the chain of elements portrayed in FIG. 13a.

FIG. 13c illustrates yet another embodiment of a chain of elements suitable for use as filling for an intermediary support structure inside a wrapping in accordance with the present invention. Here the cylinders 148 are linked to each other by means of a string or strings 146.

The shape of the elements may vary and in fact any bulky shape should suffice.

The linked elements are provided in a linked configuration for ease in their deployment, but the links are preferably loose so as to allow detachment and redistribution of the elements in a free manner. Some of the elements remain linked and this contributes to the overall stability, whereas some elements are disconnected and act separately occupying the space within the wrapping optimally.

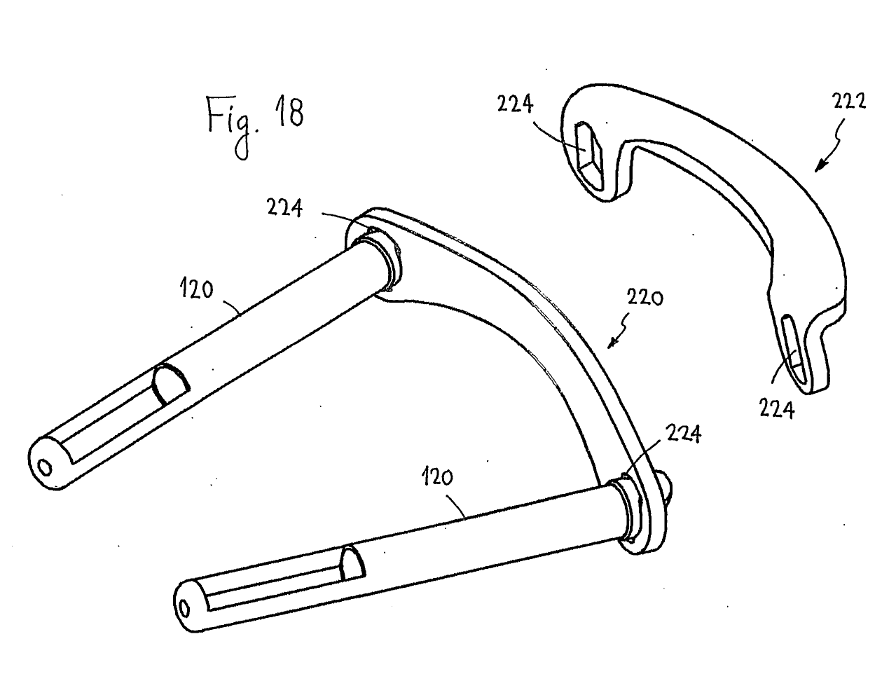

The feeding of the plurality of elements for the expandable structure may be done using a cartridge filled with these elements, using chains of linked elements or even feeding it manually. In order to enhance their compactness, a pressing element may be used, such as a pressing rod used with or without power-multiplying device (see FIG. 18 for an example).

The plurality of elements may also be initially placed within the introduction component, so that when a pressure is applied through the conduit, some of the elements are forced out through the slits to the desired height.

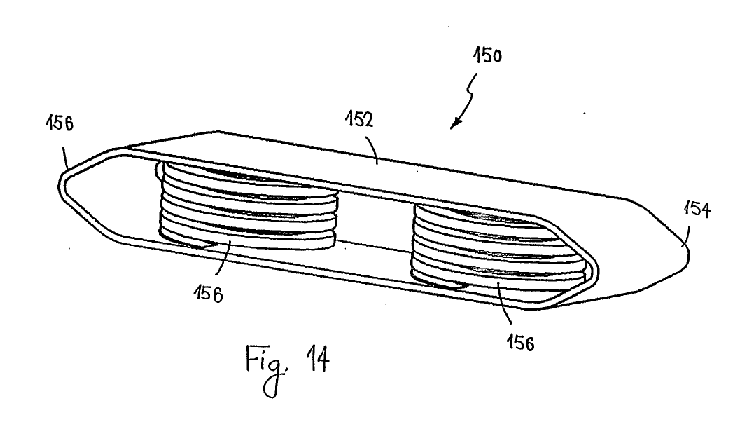

FIG. 14 illustrates an embodiment of a shock absorbing support element. This particular embodiment of a support structure may be used as intermediary support structure as explained hereinabove. It may also be suitable for use in connection with a vertebral modular distraction device such as described in PCT/IL02/00916, or by Johnson (US 2002/0183761). The shock absorbing support element 150 is made of a strap 152 of elastic material, such as metal for example, shaped in the form of a bar with pointed wedge-like ends 154. Within the bar one or more springs 156 are positioned so as to enhance the elasticity of the bar. The support structure is designed to act as an adjustable suspension/shock absorber device (e.g. to prevent adjacent vertebra fracture)

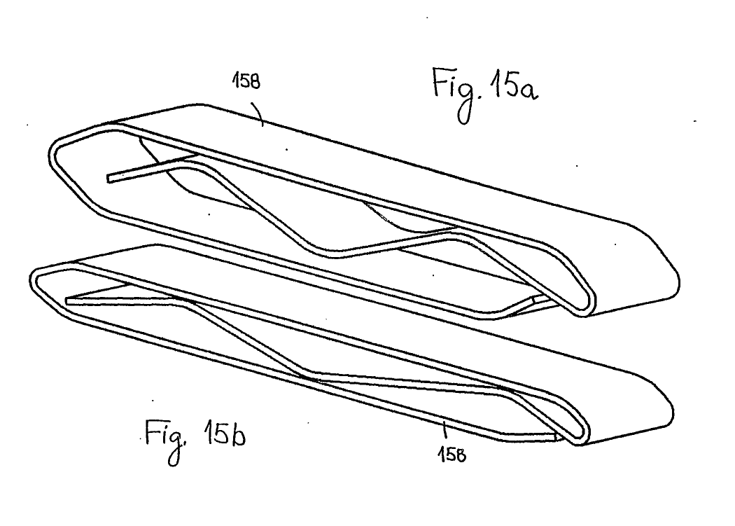

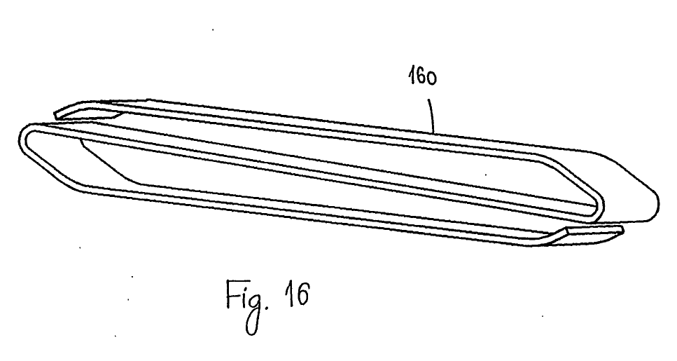

FIG. 15a illustrates another embodiment of a shock absorbing support element. The support element is made of a single strap 158 of a resilient elastic material that is shaped in the form of a bar with pointed wedge-like ends and includes internal wavey end that serves as a spring. FIG. 15b illustrates the resilient support element of FIG. 15a in a compressed state.

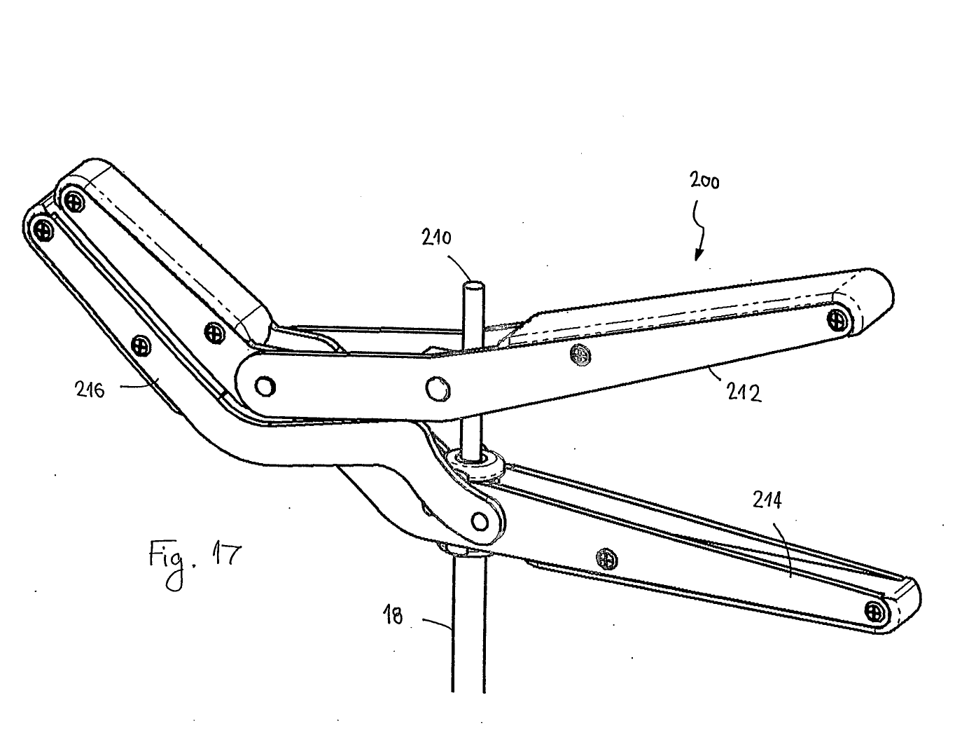

FIG. 16 illustrates yet another embodiment of a shock absorbing support element. Here too the support element is made of a single strap 158 of a resilient elastic material that is shaped in the form of a bar with pointed wedge-like ends. Extension of the strap is provided within the external surfaces, acting as a shock absorbing member to enhance the elasticity of the element.

The support elements discussed hereinabove may be used in conjunction with a vertebral modular distraction device as described in PCT/IL02/00916.

The method described in PCT/IL02/00916 for vertebral reconstruction and support comprises a minimally invasive surgical method, involving inserting support elements (or plates), through a small incision in the skin and surrounding muscle tissue, using low profile (i.e. narrow) delivery tools, into the vertebral body or into the inter-vertebral disk area, in order to reconstruct the original anatomic structures. The method fits in particular the treatment of collapsed vertebral body or degenerative disk space. After using it for reconstruction of the anatomical structure of the vertebral body, this assembly further functions as a prosthesis, which supports the vertebra internally (within the cortex) or externally (intervertebrally), substantially maintaining the normal original shape of the vertebra and the spinal structure.

A typical vertebral modular distraction system comprises a plurality of plates, capable of being mounted one on top of the other or next to each other in a lateral adjacent configuration and staying secured in that position so as to present a modular scaffolding structure.

The shape of these plates is designed to allow precise sliding of every plate on top, bellow, or next to the other. In a preferred embodiment of the present invention, in order to accomplish that aim, a recess and corresponding protrusion design is used. It is very desirable that the plate design ensures the prevention or substantial restraining of the plates from sliding off each other.

In order to place each of the plates in the desired position and location a preferable delivery system is used.

An introduction device that may be used in conjunction with the embodiments described herein was described in PCT/IL02/00916, and is incorporated herein by reference. Other introducing device suitable (although somewhat less appealing due to its single-direction nature is the device disclosed in US 2002/0183761, incorporated herein by reference.

The devices in accordance with the present invention may be used as permanent implant devices or may be used for surgical purposes and them removed from the patient's body.

The distraction device of the present invention may be used in surgery or other medical procedures since it is simple, provides a good distraction solution, and the deployed and folding of the device are relatively fast. It is noted that the device is not necessarily implantable and may be used as a temporary instrument to be removed when distraction is no longer necessary.

The embodiments disclosed herein allow for the conduit (or part of it) to detach once the device is positioned in place, if the device is to remain in position (implanted).

Some of the designs allow the folding back of the expandable element. This is of real importance since it allows the surgeon for maximal reconsideration (up to a complete removal of the device).

It is emphasized that the device disclosed in the present invention may be used intervertebrally or intravertebrally, but is not solely intended for that use, which was illustrated herein as mere example only. The device of the present invention may be used to distract and support any tissue surfaces, such as the tibial plateau, radius, and other tissues to be distracted during surgery or other medical procedures.

FIG. 17 illustrates a holder for accurate deployment of the distraction device in accordance with the present invention.

The holder 200 comprises three arms (212, 214, 216), two of which (216, 212) are pivotally connected to the third arm 214, at different locations. The arms curvatures are aimed at providing support points between the arms so as to assist in slow yet accurate introduction of an internal rod 210 (such as a pushing rod) into conduit 18. This is an optional arrangement for manipulation of the distraction device (or in fact any other device) introduced through a conduit towards and out of the distal end of the conduit, from the proximal end of the conduit. The proximal end of the conduit is fixed to arm 214, while rod 210 is fixed to arm 212. By gripping these arms and drawing them closer together the rod advances further into the conduit.

FIG. 18 illustrates a fixator 220 for fixing in position two introduction devices in accordance with the present invention in deployment. The fixator is basically a bar with a predetermined curvature, provided with two holes 224 each hole matching the profile of the proximal end of an introduction device of the distraction device 120, the fixator is used to provide additional stability to the introduction devices when two are inserted into a vertebra or into other required position to distract two opposing tissues. Fixator 222, with a slightly different profile is shown detached from introduction devices. In principle the holder can be designed with any profile that agrees with its designated task of holding the introduction devices in their desired position. A curvature is needed when considering a holder for vertebral distraction devices.

The expandable structure of the device of the present invention may be made from biodegradable materials so that over a period of time it disintegrates and dissolves.

The device of the present invention may be made from various materials, such as titanium, titanium alloy, stainless steel alloys, steel 316, processed foil, hydroxyapatite, material coated with hydroxyapetite, plastics, silicon, composite materials, carbon-fiber, hardened polymeric materials, polymethylmetacrylate (PMMA), ceramic materials, coral material or a combination thereof. Other materials may be suited too.

It should be clear that the description of the embodiments and attached Figures set forth in this specification serves only for a better understanding of the invention, without limiting its scope.

It should also be clear that a person skilled in the art, after reading the present specification could make adjustments or amendments to the attached Figures and above described embodiments that would still be covered by the following Claims and their equivalents.

1. A device for distracting and supporting two substantially opposing tissue surfaces in a patient's body, to be introduced within the tissue surfaces in a minimally invasive procedure, the device comprising:
a wrapping element; and
an expandable structure insertable between the two support surfaces, adapted to be expanded between the two support surfaces to a predetermined dimension.

2. The device as claimed in claim 1, further comprising a conduit, through which it is introduced into the patient's body.

3. The device as claimed in claim 1, further comprising with a introduction member, the introduction member comprising a substantially linear conduit, having a proximal end through which the device is inserted and a distal end where two substantially opposite slots are provided, through which the expandable structure may protrude in directions substantially perpendicular to the conduit.

4. The device as claimed in claim 3, wherein the wrapping element comprises an adjustable strap interlaced through slits that are provided on the introduction member.

5. The device as claimed in claim 1, wherein the wrapping element comprises two substantially opposing support surfaces.

6. The device as claimed in claim 5, wherein the two substantially opposing support surfaces are ragged on internal sides.

7. The device as claimed in claim 6, wherein at least one of the two substantially opposing support surfaces is provided with a protrusion for providing anchorage for the expandable structure when it is positioned between the two substantially opposing support surfaces.

8. The device as claimed in claim 6, wherein the expandable structure comprises a plurality of beams.

9. The device as claimed in claim 1, wherein the expandable structure comprises a segmented strip made of a series of jointed segments pivotally interconnected so as to present a multi-joint strip, each segment having an elongated bore provided on it through which a fastener may be interlaced, for holding the strip in a folded state of a desired height.

10. A device for distracting and supporting two substantially opposing tissue surfaces in a patient's body, to be introduced within the tissue surfaces in a minimally invasive procedure, the device comprising:
a segmented strip made of a series of jointed segments pivotally interconnected so as to present a multi-joint strip, each segment having an elongated bore provided therewithin, through which a fastener may be interlaced, for holding the strip in a folded state of a desired height.

11. The device as claimed in claim 1, wherein the expandable structure is an initially collapsed deployable polyhedron structure.

12. The device as claimed in claim 11, wherein the polyhedron structure has a cross section in the form of a parallelogram.

13. (canceled)

14. The device as claimed in claim 1, wherein the expandable structure comprises two foldable straps placed on either sides of a bar.

15. The device as claimed in claim 1, wherein the expandable structure comprises a coil.

16. The device as claimed in claim 15 wherein the coil comprises a coiled strap.

17. The device as claimed in claim 16, further comprising a harness arrangement with two substantially parallel bars pivotally connected to an introducing conduit and coupled to an axle for the strap to be coiled on, allowing upward or downward motion of the coil with respect to the conduit.

18. The device as claimed in claim 16, wherein the coiled strap is coiled over a rotor.

19. The device as claimed in claim 16, wherein the device further comprises a propulsion belt for driving the strap and enhancing its coiling.

20. The device as claimed in claim 19, further comprising with a roller for rolling the propulsion belt.

21. The device as claimed in claim 19, wherein the belt comprises a ragged surface for enhancing friction between the belt and the coil.

22. The device as claimed in claim 19, wherein the strap further comprises a ragged surface for enhancing friction between the belt and the coil.

23. The device as claimed in claim 1, wherein the expandable structure comprises a plurality of cylindrical elements.

24. The device as claimed in claim 23, wherein the cylindrical elements are provided with cog-like surface.

25. The device as claimed in claim 23, wherein the cylindrical elements are provided with threading.

26. The device as claimed in claim 23, wherein the cylindrical elements are linked.

27. The device as claimed in claim 26, wherein the cylindrical elements are linked loosely by links that can break up when the linked cylindrical elements are pressed within the wrapping element.

28. The device as claimed in claim 26, wherein the cylindrical elements are linked by a string.

29. The device as claimed in claim 1, wherein the wrapping element is incorporated with the expandable structure.

30. The device as claimed in claim 1, wherein the wrapping is incorporated with an introduction device used to introduce the device to a target location.

31. The device as claimed in claim 1, made form materials selected from: metal, titanium, titanium alloy, stainless steel alloys, steel 316, processed foil, hydroxyapatite, material coated with hydroxyapetite, plastics, silicon, composite materials, carbon-fiber, hardened polymeric materials, polymethylmetacrylate (PMMA), ceramic materials, coral material or a combination thereof.

32. A plate for use in an assembly for distracting and supporting two opposing tissues, the assembly comprising at least one of a plurality of plates, the plate comprising a flexible structure made in the form of a strap.

33. The plate as claimed in claim 32, wherein the strap forms a structure having substantially two opposing surfaces and a portion of the strap in between the surfaces in a wavy formation.

34. The plate as claimed in claim 33, wherein portions of the strap form wedge-like ends located on either sides of the substantially opposing surfaces.

35. (canceled)
